SEQUENCE LISTING (20 sequences)

SEQ ID NO: 1 - DNA, 20 bp, artificial sequence
  primer
ggctgtattcccctccatcg

SEQ ID NO: 2 - DNA, 22 bp, artificial sequence
  primer
ccagttggtaacaatgccatgt

SEQ ID NO: 3 - DNA, 19 bp, artificial sequence
  primer
atgttgcggtcgctacgtc

SEQ ID NO: 4 - DNA, 17 bp, artificial sequence
  primer
agaagttgccaccgccg

SEQ ID NO: 5 - DNA, 16 bp, artificial sequence
  primer
cgatgccctcaacgcc

SEQ ID NO: 6 - DNA, 24 bp, artificial sequence
  primer
cttagggttactgtagccgtaggc

SEQ ID NO: 7 - DNA, 19 bp, artificial sequence
  primer
atgttgcggtcgctacgtc

SEQ ID NO: 8 - DNA, 17 bp, artificial sequence
  primer
agaagttgccaccgccg

SEQ ID NO: 9 - DNA, 20 bp, artificial sequence
  primer
cgtaccctgacaccaatctc

SEQ ID NO: 10 - DNA, 21 bp, artificial sequence
  primer
ctcctcttcgcacttctgctc

SEQ ID NO: 11 - DNA, 20 bp, artificial sequence
  primer
ggtctcaacccccagctagt

SEQ ID NO: 12 - DNA, 23 bp, artificial sequence
  primer
gccgatgatctctctcaagtgat

SEQ ID NO: 13 - DNA, 23 bp, artificial sequence
  primer
atgagtttttcccttatggggac

SEQ ID NO: 14 - DNA, 21 bp, artificial sequence
  primer
gctggaagttggacacctcaa

SEQ ID NO: 15 - DNA, 21 bp, artificial sequence
  primer
tttaactcccttggcgcaaaa

SEQ ID NO: 16 - DNA, 20 bp, artificial sequence
  primer
ctttccctccgcattgacac

SEQ ID NO: 17 - DNA, 23 bp, artificial sequence
  primer
tagtccttcctaccccaatttcc

SEQ ID NO: 18 - DNA, 21 bp, artificial sequence
  primer
ttggtccttagccactccttc

SEQ ID NO: 19 - DNA, 21 bp, artificial sequence
  primer
atgaacgctacacactgcatc

SEQ ID NO: 20 - DNA, 21 bp, artificial sequence
  primer
ccatccttttgccagttcctc

(19) United States
(12) Patent Application Publication
(10) Pub. No.: US 2016/0209425 A1
(43) Pub. Date: Jul. 21, 2016

(54) USE OF DECTIN-1 ACTIVATORS FOR TREATMENT OF LIVER DISORDERS

(71) Applicant: NEW YORK UNIVERSITY, NEW YORK, NY (US)

(72) Inventors: GEORGE MILLER, ENGLEWOOD, NJ (US); LENA SEIFERT, NEW YORK, NY (US)

(21) Appl. No.: 15/000,435

(22) Filed: Jan. 19, 2016

Related U.S. Application Data
(60) Provisional application No. 62/104,217, filed on Jan. 16, 2015.

(51) Int. Cl. G01N 33/68; C07K 16/28; C12Q 1/68; A61K 38/17

(52) U.S. Cl. CPC G01N 33/6872; A61K 38/1709; C07K 16/2896; C12Q 1/6883; C07K 2317/76; G01N 2333/705; G01N 2333/70596; C12Q 2600/136; C12Q 2600/158; G01N 2500/04; G01N 2500/10

(57) ABSTRACT

Provided is a method of treatment of liver disorders comprising administrating to an individual in need of treatment, a therapeutically effective amount of an activator of Dectin-1 pathway. Examples of activators of Dectin-1 pathway include Dectin-1 ligands. Also provided is a method of identifying activators of Dectin-1 pathway by determining the effects of test agents on Dectin-1 ligation and events downstream of the ligation in the Dectin-1 activation pathway.

### CROSS-REFERENCE TO RELATED APPLICATIONS

This application claims priority to U.S. provisional patent application No. 62/104,217 filed on Jan. 16, 2015, the disclosure of which is incorporated herein by reference.

### STATEMENT REGARDING FEDERALLY-SPONSORED RESEARCH

This invention was made with government support under grant number DK085278 from the National Institutes of Health. The government has certain rights in the invention.

### BACKGROUND OF THE DISCLOSURE

Hepatic fibrosis—the end result of repeated liver injury—is one of the most significant public health concerns worldwide. Liver injury resulting from a variety of etiologies including viral hepatitis, toxins, or metabolic disorders primes hepatocytes to regenerate to replace necrotic or apoptotic hepatic parenchymal cells and simultaneously triggers a robust inflammatory response that induces hepatic stellate cells (HSC) to transdifferentiate and express extracellular matrix (ECM) protein. If injury persists, regeneration eventually fails, and the hepatocytes are replaced by abundant ECM leading to fibrosis and eventually cirrhosis. Moreover, liver fibrosis strongly predisposes to hepatocyte transformation and the development of hepatocellular carcinoma (HCC), the 3^{rd} leading cause of cancer-related death worldwide.

Toll-like receptor (TLR) ligation is a primary mechanism by which intra-hepatic innate inflammatory cells and HSC are activated after hepatic injury. TLRs belong to a broader category of evolutionarily conserved pattern recognition receptors (PRRs), which link inflammatory responses to pathogenic or sterile inflammatory stimuli. TLR ligation is considered to have a critical role in perpetuating sterile inflammation and tissue damage in chronic liver disease. Similarly, TLR4 ligation by lipopolysachharide (LPS) derived from selected intestinal microbiota is known to promote hepatocellular carcinogenesis.

Dectin-1 is a member of the C-type lectin family of pattern recognition receptors (PRRs) and is required for inflammatory responses to fungal pathogens. However, Dectin-1 is not known to be physiologically relevant in liver function.

### SUMMARY OF THE DISCLOSURE

This disclosure is based on our findings that Dectin-1 deletion markedly accelerates hepatic fibrosis and hepatocellular tumorigenesis. Further, our mechanistic work indicates that Dectin-1 protects against liver fibrosis by negatively regulating TLR4 activation by directly mitigating expression of TLR4 and its co-receptor CD14 as well as inducing over-expression of diverse signaling mechanisms which can suppress activation of NF-κB. We show that Dectin-1 modulation of CD14 expression on hepatic innate inflammatory cells is contingent on M-CSF. This is the first identification of a role for Dectin-1 in non-pathogen-driven sterile inflammation and provides support for targeting Dectin-1 for therapeutics in hepatic fibrosis and HCC. Moreover, our work has pleiotropic implications for understanding reciprocal regulation between families of PRRs which is critical for maintaining physiologic homeostasis in health and disease. The results of the present disclosure are applicable for mitigating liver fibrosis, and hepatocarcinogenesis, as well as in other sterile inflammatory processes and inflammation-driven cancers.

The disclosure provides a method for identifying an agent which can activate Dectin-1 signaling pathway in liver leukocytes. The method comprises testing one or more test agents for their ability to activate Dectin-1 signaling pathway. In one embodiment, an agent or a plurality of agents may be tested simultaneously. Identification of Dectin-1 agonists may be carried out by determining the effect of test agents on one or more of the markers, pathways, signals etc. disclosed herein as being relevant to the action of Dectin-1.

In one aspect, this disclosure provides a method for treatment of liver related disorders comprising activating Dectin-1 signaling pathway. For example, the method can comprise administration of one or more activators of Dectin-1 signaling pathway for treatment of liver related disorders. Dectin-1 agonists may be used for reducing the severity of or treating the symptoms of liver fibrosis and/or liver cancer. In one embodiment, Dectin-1 or its agonists may be used for liver regeneration.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. High Dectin-1 Expression in Hepatic Leukocytes and in Liver Fibrosis in Mice and Humans. (A) Liver non-parenchymal cell (NPC) suspensions from WT mice were tested for co-expression of Dectin-1 and CD68 by confocal microscopy (scale bar, 10 mm). Similarly, Dectin-1 expression was tested in murine WT and Dectin-1−/− fibrotic liver CD45+ pan-leukocytes, DC (CD11c+MHCII+), and macrophages (CD11c-Gr1-F480+CD11b+) compared with their counterparts in spleen by flow cytometry. Gray histograms represent isotype controls. Representative data and summary data from five separate experiments are shown. (B) Human liver NPC were tested for co-expression of Dectin-1 and CD11b by confocal microscopy (scale bar, 10 mm). The fraction of Dectin-1+ cells among CD45+ pan-leukocytes and CD14+ monocytes was compared in human liver versus PBMC by flow cytometry. Representative histograms and summary data are shown. (C) Dectin-1 expression was tested in cultured murine hepatic stellate cells by both confocal microscopy and flow cytometry compared with Dectin-1^{-/-} HSC and isotype control (scale bar, 10 mm). (D) Murine CD45-CD146+ liver sinusoidal endothelial cells (LSECs) and CD45−CD146− hepatocytes derived from fibrotic liver were tested for expression of Dectin-1 compared with isotype control (gray histogram). (E and F) Expression of Dectin-1 was tested in CD11c+MHCII+ DC (E) and CD11c-Gr1-F480+CD11b+ (F) macrophages from control liver versus liver from mice with TAA-induced liver fibrosis compared with isotype control. Representative data and summary data from three experiments are shown. (G and H) Normal and fibrotic murine (G) and human liver (H) were tested for infiltration of Dectin-1+ cells by immunohistochemistry compared with isotype control (scale bar, 100 mm). Each experiment was repeated at least three times with similar results (*p<0.05, **p<0.01, ***p<0.001,****p<0.0001).

FIG. 2. Dectin-1 Deletion Exacerbates Hepatic Fibrosis WT and Dectin-1−/− mice were treated with TAA for 12 weeks. (A) Livers were harvested and analyzed by H&E, Trichrome, and Sirius Red staining (scale bar, 1 mm). (B) Fibrosis was quantified based on Trichrome staining using a computerized grid. (C-F) Serum levels of ALT and AST (D) were analyzed. Liver tissues were tested for (E) expression of TIMP2, TIMP4, MMP7, and MMP2 by western blotting and for (F) expression of Plau, Thbs1, Serpine1, and Mmp9 by RT-PCR. (G and H) HSC activation in TAA-treated WT and Dectin-1−/− liver was tested by expression of a-SMA (scale bar, 100 mm) (G), and hepatocyte proliferation was assessed using PCNA immunohistochemistry (H) (n=20 mice/group; *p<0.05, **p<0.01).

FIG. 3. Dectin-1 Regulates Intra-Hepatic Inflammation in Fibrotic Liver (A-C) WT and Dectin-1−/− mice were treated with TAA for 12 weeks. Livers were harvested and analyzed for infiltration with (A) CD45+ pan-leukocytes, (B) MPO+ neutrophils, and (C) CD68+ macrophages (scale bar, 100 mm) by immunohistochemistry. Representative images and summary data are shown. (D) Serum MCP-1 levels were measured in control and fibrotic WT and Dectin-1−/− liver. (E) The fraction of DC in hepatic NPC suspensions was compared in PBS- and TAA-treated WT and Dectin-1−/− liver. (F and G) Liver MHC 11+ APC from control and TAAtreated WT and Dectin-1−/− liver were tested for (F) expression of IL-6 and (G) TNF-a using intracellular cytokine analysis. Each experiment was repeated more than three times with similar results (n=6 mice/group; **p<0.01).

FIG. 4. Dectin-1 Deletion Promotes Hepatocarcinogenesis (A) Dectin-1 expression was tested by immunohistochemistry in human HCC. Representative interstitial and tumor cell-rich areas are shown (scale bar, 100 mm). (B) Tumor nodules were digested with collagenase, and CD45− cells were tested for Dectin-1 expression compared with isotype control. (C) Dectin-1 expression was compared in CD11c+MHCII+ DC and Gr1-CD11c-F4/80+CD11b+ macrophages from control liver versus HCC nodules in mice. Representative data and summary data from three animals are shown. (D) WT and Dectin-1−/− mice were treated with CC14+DEN. Representative images of mice and liver tumors at 24 weeks are shown. The percentage of liver weight per body weight, the maximal diameter of the largest tumor in each animal, and the number of surface liver tumors per mouse were calculated. (E) Tumors were tested for expression of NF-kB and MAP Kinase signaling intermediates, MyD88, and TRAF6 by western blotting. Representative data and density plots based on triplicates are shown. (F) Tumors were tested for oncogenic genes using a RT-PCR array (n=6 mice/group; *p<0.05, **p<0.01).

FIG. 5. Dectin-1 Negatively Regulates TLR4 Activation (A) WT and Dectin-1−/− mice were treated for 12 weeks with a TLR4 inhibitor alone, TAA alone, or both in combination (n=5/group). Trichrome staining of representative liver sections is shown, and the fraction of fibrotic area was calculated for each cohort using a computerized grid (scale bar, 10 mm). (B-D) WT and Dectin-1−/− mice were treated with LPS and assayed for (B) serum levels of IL-6, (C) core body temperature at serial time intervals, and (D) survival (n=6 mice/group). (E and F) Splenocyte suspensions derived from WT and Dectin-1−/− mice were treated in vitro with PBS or LPS and tested for both (E) TNF-a production in cell-culture supernatant and (F) cellular proliferation. (G-I) HSCs derived from WT and Dectin-1−/− mice were treated in vitro with PBS or LPS and tested for production of (G) TNF-a, (H) IL-6, and (I) MCP-1 in cell-culture supernatant. (J) Fibrotic Dectin-1−/− and WT liver tissue were assayed for expression of activated signaling intermediates downstream of TLR4 ligation by western blotting. Representative data and density plots based on quadruplicates are shown. In vitro experiments were repeated at least three times using three to five mice per experimental group (*p<0.05, **p<0.01, ***p<0.001).

FIG. 6. Dectin-1 Suppresses TLR4 and CD14 Expression, which Mediates Dectin-1 Regulation of Liver Fibrosis and Inflammation (A and B) WT and Dectin-1−/− mice were treated for 12 weeks with PBS or TAA. CD11c-Gr1 F480+CD11b+ macrophages were tested for (A) TLR4 and (B) CD14 expression by flow cytometry. Representative data and summary data from five mice per group are shown. (C and D) Macrophages were stimulated in vitro for 24 hr with an array of Dectin-1 ligands and then tested for expression of (C) TLR4 and (D) CD14. (E) WT mice were challenged in vivo with LPS alone or in combination with an array of selective Dectin-1 ligands or vehicle. Core body temperature was measured at 12 hr. (F) WT mice were challenged in vivo with LPS alone or LPS+Curdlan. Serum cytokines were measured at 12 hr (error bar, 1 mm). (G and H) WT mice were treated for 12 weeks with PBS, depleted Zymosan alone, TAA alone, or depleted Zymosan+TAA. Trichrome staining of representative liver sections are shown, and the fraction of fibrotic area was calculated for each cohort using a computerized grid. (H) Serum levels of MCP-1 were calculated (n=5/group). (I and J) WT and Dectin-1−/− mice were challenged in vivo with LPS alone or LPS+CD14 blockade (n=5/group). Effects of CD14 inhibition on (I) lowering serum levels of TNF-a and IL-6 and (J) increasing core body temperature compared with LPS treatment alone are shown (error bar, 1 mm). (K) TAA-treated WT and Dectin-1−/− mice were administered isotype or a neutralizing a-CD14 mAb during the 12-week course of fibrosis induction. Trichrome staining of representative liver sections are shown, and the fraction of fibrotic area was calculated for each cohort using a computerized grid (n=4/group; *p<0.05, **p<0.01, ***p<0.001).

FIG. 7. M-CSF Blockade Lowers CD14 Expression and Protects against Fibrosis in Dectin-1−/− Liver (A) Paraffin-embedded liver sections from PB Sand TAA-treated WT and Dectin-1−/− mice were stained using a mAb specific for M-CSF. Representative images are shown (n=5; scale bar, 100 mm). (B) WT mice were challenged in vivo with LPS alone or in combination with an array of selective Dectin-1 ligands or vehicle. Serum M-CSF was measured at 12 hr (n=3 mice/group). (C) WT and Dectin-1−/− mice were treated with TAA for 12 weeks. Selected cohorts were additionally treated with a-M-CSF or isotype control. DC and macrophage subsets were gated by flow cytometry and tested for expression of CD14. Representative data from one liver and summary data from five mice are shown (error bar, 1 mm). (D) WT and Dectin-1−/− mice were treated with PBS or TAA for 12 weeks. Select cohorts were additionally treated with a-M-CSF or isotype control. Liver sections were stained with Trichrome, and fibrosis was quantified using a computerized grid. (E and F) Macrophages were treated with PBS or recombinant M-CSF and tested for (E) CD14 expression by flow cytometry and (F) TNF-a and MCP-1 expression in cell-culture supernatant. (G and H) WT mice were co-administered LPS and M-CSF alone or in combination. (G) Serum levels of IL-6 and (H) core body temperature were measured (n=5/group; *p<0.05, **p<0.01, ***p<0.001).

FIG. 8. WT and Dectin-1−/− liver have similar phenotypes at baseline. (A) Paraffin-embedded liver sections from PBS-treated WT and Dectin-1−/− mice were stained using Trichrome and (bar=1 mm) (B) using a mAb directed against CD45 (bar=10 μm). (C) Serum levels select immune-modulatory cytokines were measured. (D) Liver tissue derived from PBS-treated WT and Dectin-1−/− mice were tested by RT-PCR for expression of select cytokines, chemokines, and chemokine receptors. (E) Expression of inflammatory signaling intermediates and ECM proteins were tested in PBS-treated WT and Dectin-1−/− liver by western blotting. Representative data and density analysis of triplicates are shown.

FIG. 9. Dectin-1 deletion exacerbates hepatic fibroinflammatory disease in a CC14 model whereas Mincle deletion does not alter severity of liver fibrosis. (A-D) WT and Dectin-1−/− mice were treated with CC14 for 12 weeks (bar=100 μm). (A) Livers were harvested and analyzed by H&E, Trichrome staining, and using a mAb directed against CD45. (B) Fibrosis was quantified based on Trichrome staining. (C) Serum ALT and AST were measured. (D) The volume of CD45+ leukocytic infiltrate was calculated by examining 10 HPFs per liver (n=5 mice/group; *p<0.01; bar=100 μm). (E-G) WT and Mincle−/− mice were treated with TAA for 12 weeks. (E) Livers were harvested and analyzed by Trichrome staining and using a mAb directed against CD45. (F) Fibrosis was quantified based on Trichrome staining and (G) the volume of CD45+ leukocytic infiltrate was calculated by examining 10 HPFs per liver (n=5 mice/group).

FIG. 10. Dectin-1 deletion augments intra-hepatic inflammation in chronic liver disease. Fibrotic WT and Dectin-1−/− liver were tested using a nanostring assay for mRNA levels of (A) complement-related genes, (B) chemokines, (C) chemokine receptors, and (D) IL-1, IL-6, and TGF-β related genes (n=3/group). (E) CD45.1 mice were irradiated (950 Rad) and made chimeric using bone marrow derived from CD45.2 WT mice. At seven weeks splenocytes were harvested from chimeric mice and untreated CD45.1 controls and tested for expression of CD45.1 and CD45.2. Representative flow cytometry data is shown (n=3; bar=100 μm). (F) WT and Dectin-1−/− mice were irradiated and made chimeric using either WT and Dectin-1−/− bone marrow before inducing TAA-mediated liver fibrosis 7 weeks later. Livers were harvested after 12 weeks and stained using Trichrome (n=5/group; *p<0.05).

FIG. 11. Dectin-1 and TLR4 co-associate in liver inflammatory cells. (A) We precipitated TLR4, Dectin-1 or control from the lysate of murine liver NPC derived from WT, Dectin-1−/−, and TLR4−/− mice, respectively, and probed using an anti-Dectin-1 mAb suggesting that TLR4 and Dectin-1 co-associate. (B) We precipitated TLR4, Dectin-1 or control mAb from the lysate of murine liver NPC derived from WT mice and probed using anti-TLR4 again suggesting that TLR4 and Dectin-1 co-associate. (C) Murine liver NPC from WT and Dectin-1−/− mice were co-stained using mAbs directed against TLR4, Dectin-1, or isotype controls and imaged by confocal microscopy (bar=10 m). Experiments were repeated 3 times. Results were quantified by examining 10 HFPs per slide.

FIG. 12. MyD88 inhibition protects against liver fibrosis in WT and Dectin-1−/− mice. TAA-treated WT and Dectin-1−/− mice were serially administered MyD88 inhibitory peptide or a control peptide. Trichrome staining of representative liver sections are shown and the fraction of fibrotic area was calculated (n=4-5/group; **p<0.01; bar=100 m).

FIG. 13. Dectin-1 regulates CD14 expression in inflammatory and neoplastic disease and mediates differential effects of CD14 blockade in TLR4-activated leukocytes and HSC. (A) Cd14 expression was tested by RT-PCR in normal and fibrotic liver from WT and Dectin-1−/− mice (n=5/group). (B) Cd14 expression was tested by RT-PCR in HCC tumor nodules in WT and Dectin-1−/− liver (n=6/group) (C, D) WT and Dectin-1−/− mice were treated for 12 weeks with PBS or TAA. (C) CD11c-Gr1-F480+ cells were gated and tested for co-expression of CD11b and TLR2. (D) Similarly CD11c+ cells were gated and tested for co-expression of MHCII and TLR2. (E, F) PBS-treated WT and Dectin-1−/− mice were administered a neutralizing CD14 mAb or isotype control and tested for (E) changes in core body temperature and (F) serum levels of inflammatory cytokines (n=3 mice/group; p=ns for all comparisons). (G) TAA-treated WT and Dectin-1−/− mice were administered a neutralizing α-CD14 mAb or isotype control during the 12 week course of fibrosis induction. MHCII+ liver APC from each cohort were tested for expression of TNF-α by flow cytometry (n=4/group). Representative data and summary data are shown (*p<0.05, **p<0.01). (H) WT and Dectin-1−/− HSC were treated in vitro with LPS alone or LPS+CD14 blockade. Cells were harvested at 24 h and tested for expression of pPDGFRPβ, MMP7, MMP9, TIMP2, and β-actin.

FIG. 14. M-CSF blockade differentially suppresses TLR4-activated Dectin-1−/− leukocytes. (A) WT and Dectin-1−/− HSC were stained using a mAb directed against the M-CSF receptor CD115. (B) Macrophages from WT and Dectin-1−/− liver were treated with PBS or LPS and tested for co-expression of CD14 and CD115. (C) PKC activity was tested in LPS-treated WT and Dectin-1−/− HSC. (D) WT and Dectin-1−/− HSC were stimulated with LPS+vehicle or LPS+PKC inhibitor GF109203X. M-CSF was tested in cell culture supernatant at 24 h. (E) CD14 expression was measured in splenic macrophages treated with PBS, LPS alone, anti-M-CSF mAb alone, or LPS+anti-M-CSF mAb. Representative data and summary data are shown. (F) Macrophages were treated with PBS, LPS, or anti-TNF-α, alone or in combination. CD14 expression was tested by flow cytometry at 24 h. All experiments were performed in triplicate and repeated at least twice (*p<0.05, **p<0.01, ***p<0.001).

FIG. 15. γδT cells expand and activate after partial hepatectomy and are necessary for robust liver regeneration. (A) Changes in the number of γδT cells in the remnant liver after partial hepatectomy compared with sham laparotomy were tested at 3, 6, and 24 hours. (B) Expression of CCL20 was compared at baseline and at 3 h after partial hepatectomy by multi-color immune-fluorescence microscopy. (C) Hepatic CD3⁺TCRγδ⁺ cells from partial hepatectomy and sham-operated liver were gated and tested for expression of CD44, CD54, and Vγ1.1⁺ at 3 h. A representative experiment and averages of 3 replicates are shown. (D) Livers from WT and TCRδ^{-/-} mice were harvested after 70% hepatectomy and tested for nuclear expression of PCNA, Ki67, and BrdU. Representative stained sections from the 36 h time point are shown (n=5 mice/time point). (E) The relative increase in expression of c-fos, c-jun, and c-myc at 1 h in hepatectomy-treated WT and TCRδ^{-/-} livers compared with sham laparotomy-treated controls was tested by PCR. In addition, Cyclin D1 mRNA expression was tested at various time points in partial hepatectomy and sham-treated WT and TCRδ^{-/-} livers. (F) Hepatic expression of Cyclin D1 and HGF were assayed at 3 h by Western blotting. Each experiment was repeated at least 3 times (*p<0.05, **p<0.01, ***p<0.001).

FIG. 16. γδT cell depletion diminishes expression of pro-regenerative inflammatory mediators and modulates leukocyte expansion in the regenerating liver. (A) Expression of p-STAT3, STAT, p-p38, p-Erk1/2, Erk1/2, Notch-1, and β-actin were tested by Western blotting within 3 h after surgery in partial hepatectomy and sham-laparotomy treated WT and TCRδ^{-/-} mice. (B) Hepatic expression of IL-4, IL-6, IL-22, and IL-17 were tested in hepatectomized WT and TCRδ^{-/-} mice at 1 and 3 hours and compared with sham-laparotomy controls by PCR. (C) Serum IL-6 was measured in WT and TCRδ^{-/-} mice at 48 h after partial hepatectomy. (d) IFNγ expression was compared at 3 h in regenerating WT and TCRδ^{-/-} liver by PCR. (E) WT and TCRδ^{-/-} mice were sacrificed 36 h after partial hepatectomy. Liver sections were stained using antibodies directed against CD45 (40×), B220 (40×), MPO (40×), and CD68 (20×). The fraction of positive cells per HPF for each respective marker is shown. (F) The fraction of NK1.1⁺CD3⁻ NK cells, NK1.1⁺CD3⁺ bulk NKT cells, and NK1.1⁺CD1d tetramer⁺ iNKT cells were determined using flow cytometry in WT and TCRδ^{-/-} mice at 36 h after partial hepatectomy. Representative data and averages of 3 replicates are shown. Experiments were repeated 3 times using 3-5 mice per group (*p<0.05, ***p<0.001).

FIG. 17. γδT cells influence the pro-regenerative phenotype in hepatic inflammatory cells. (A) NK cells and (B) NKT cells harvested from the regenerating liver of WT and TCRδ^{-/-} mice at 36 h were gated and tested for expression of CD69 and IFNγ. (C) Hepatocyte proliferation was tested by expression of PCNA and Ki67 at 36 h in WT and TCRδ^{-/-} mice depleted of NK1.1⁺ cells or mock depleted (n=3/group). (D) CD11c⁻F480⁺ Kupffer cells and (E) CD11c⁺MHCII⁺ DC were gated from the regenerating liver of WT and TCRδ^{-/-} mice at 36 h and tested for expression of IL-6. Representative data and averages of replicates are shown. Each experiment was repeated at least 3 times (*p<0.05, ***p<0.001).

FIG. 18. γδT cells induce a pro-regenerative phenotype in hepatic inflammatory cells in an IL-17 dependent manner. (A) IL-17 expression was compared at 3 h by intracellular cytokine analysis in γδT cells from hepatectomy versus sham-operation liver in WT mice. Averages of quadruplicates is shown. (B) CD3⁺ T cells from regenerating WT liver were gated and tested for co-expression of TCRγδ and IL-17. Similarly, CD3⁻ whole liver cellular concentrates were analyzed using mAbs directed against CD45 and IL-17. Based on triplicate experiments, the fraction of γδT cells, αβT cells, hepatic innate inflammatory cells, and hepatic parenchymal expression of IL-17 is shown. (C) Liver NPC were harvested at 3 h from regenerating murine liver of C57BL/6-Trdc^{tm1Mal}/J mice in which the γδT cells express GFP and stained for IL-17 (red) and DAPI (blue). A representative overlay image is shown. (D, E) Leukocyte concentrates from WT and TCRδ^{-/-} mice were stimulated with PMA-Ionomycin and tested for (d) IL-6 and (e) IFNγ expression by PCR, both alone or in the context of IL-17 blockade (*p<0.05, **p<0.01). (F) Leukocyte concentrates from WT and TCRδ^{-/-} mice were stimulated with PMA-Ionomycin. NKT cells were gated and tested for IFNγ expression by intracellular cytokine staining in the context of IL-17 blockade or control mAb. Representative data and average of triplicate repeats are shown. All experiments were performed at least 3 times with similar results (*p<0.05, **p<0.01, ***p<0.001).

FIG. 19. Dectin-1 dependent IL-22 expression in γδT cells is vital for robust liver regeneration. (A) IL-22 mRNA expression was tested at 3 h in partial hepatectomy and sham-laparotomy treated liver by PCR. (B) CD3⁺ T cells from regenerating WT liver were gated and tested for co-expression of TCRγδ and IL-22. Similarly, CD3⁻ cells were gated and whole liver cellular concentrates were analyzed using mAbs directed against CD45 and IL-22. Based on quadruplicate experiments, the fraction of γδT cells, αβT cells, hepatic innate inflammatory cells, and hepatic parenchymal cell expression of IL-22 was calculated. (C) Expression of Dectin-1 in liver γδT cells was compared in partial hepatectomy and sham-laparotomy treated mice. Representative data and the change in Dectin-1 expression based on MFI in 4 replicate experiments is shown. (D) Hepatic γδT cells were tested for co-expression of IL-22 and Dectin-1 by flow cytometry and confocal microscopy. (E) IL-22 expression was tested in γδT cells from WT and Dectin-1^{-/-} mice at 3 h after hepatectomy by intracellular cytokine analysis. Summary data based on 3 replicates is shown. In addition, hepatic γδT cells were stimulated with the selective Dectin-1 ligand Zymosan depleted or saline and tested for expression of IL-17 and IL-22. (F) IL-22 was measured in the serum of WT and TCRδ^{-/-} mice at 48 h post-hepatectomy and SOCS3 mRNA expression was tested in WT and TCRδ^{-/-} mice at multiple early time points post-hepatectomy (*p<0.05, ***p<0.001).

FIG. 20. Dectin-1 mediated IL-17 cytokine production is necessary for the development of a pro-regenerative phenotype. (A, B) Leukocyte concentrates from WT and TCRδ^{-/-} mice were stimulated in vitro with the selective Dectin-1 ligand Zymosan depleted and tested for expression of (A) IL-6 mRNA or (B) protein in the context of IL-17 blockade or control mAb. (C) DC or macrophages were gated by flow cytometry in WT and TCRδ^{-/-} leukocyte suspensions and tested for expression of IL-6 by intracellular cytokine staining. (D, E) Similarly, bulk NKT cells were gated in (D) WT and (E) TCRδ^{-/-} leukocyte suspensions and tested for expression of IFNγ in the context of IL-17 blockade or control. Experiments were repeated 3 separate times with similar results. (F) Cohorts of hepatectomy-treated WT or CD45.1⁺ mice were additionally treated with Zymosan depleted and tested at 24 h for Cyclin D1 expression by PCR and hepatocyte proliferation by PCNA staining (n=4 mice/group; *p<0.05, **p<0.01, ***p<0.001).

FIG. 21. Representation of the central position of the Dectin-1-γδT cell-IL-17/IL-22 axis in promoting hepatic regeneration.

FIG. 22. Liver γδT cells exhibit a distinct immune phenotype. (A) Live mouse splenocytes or hepatic NPC were gated and tested for co-expression of CD3 and TCRγδ. (B) CD3⁺TCRγδ⁺ cells in the spleen and liver were gated and tested for expression of cell surface markers and (C) Dectin-1. (D) γδT cells were stimulated with PMA/Ionomycin and tested for intracellular expression of IL-22 and IL-17. (E) CD3⁺TCRγδ⁺ cells were gated and the faction of Vγ1.1⁺ and Vγ4⁺ γδT cells measured in the spleen and liver. Each experiment was repeated more than 3 times with similar results. Percentage of positive cells or MFIs are were calculated. Both representative data and analyses of replicates are shown (*p<0.05; **p<0.01; ***p<0.001).

FIG. 23. Human liver γδT cells are distinctly pro-inflammatory. (A) Human liver and PBMC CD3⁺TCRγδ⁺ cells were gated and (B) tested for surface marker expression. Representative dot plots and summaries of all human experiments are shown. Each line represents data collected from an individual patient. (C-E) Human liver and PBMC γδT cells and liver CD45⁺ leukocytes excluding γδT cells were tested for IL-17 and IL-22 expression after stimulation with PMA/Ionomycin. (C) Representative data from experiments repeated more than 3 times is shown. (D) Expression of IL-17 and (E) IL-22 in human liver was compared in γδT cells, αβT cells, CD45⁺CD3⁻ inflammatory cells, and CD45⁻ parenchymal cells (*p<0.05, **p<0.01).

FIG. 24. CCL-20 expression post-hepatectomy. (A) WT mice underwent sham operation or partial hepatectomy. Hepatic expression of CCL20 was tested at baseline and at 3 h by PCR. (B) Liver from mice treated by hepatectomy were harvested at 3 h and co-stained for CCL20 and CD45 by flow cytometry. Isotype control is shown (n=3-4 mice/group; *p<0.05, ***p<0.001).

FIG. 25. Delayed hepatic regeneration in TCRδ^{-/-} liver. (A) Change in liver weight at multiple time points after partial hepatectomy was tested in WT and TCRδ^{-/-} mice (n=3-4/time point). (B) Plasma levels of Prothrombin, (C) serum AST, (D) ALT, and (E) glucose levels were measured at multiple time points after partial hepatectomy in WT and TCRδ^{-/-} mice (n=6/time point; *p<0.05, **p<0.01).

FIG. 26. γδT cell depletion using bone marrow chimeric mice inhibits liver regeneration. WT mice were made chimeric using bone marrow derived from either TCRδ^{-/-} mice or WT controls. At 6 weeks, mice were subject to partial hepatectomy and liver regeneration determined by nuclear expression of PCNA at 36 h (n=4 mice/group; ***p<0.001).

FIG. 27. γδT cells induce a pro-regenerative phenotype in hepatic inflammatory cells. (A) NKT cells or (B) Kupffer cells were cultured alone or together with hepatic γδT cells and tested for surface expression of activation markers by flow cytometry. Differences in activation marker expression were determined by calculating relative MFIs in experiments repeated 3 times. (C) Kupffer cells were tested for IL-6 expression after culture alone or with selected γδT cell subpopulations. Averages of experiments repeated 3 times were calculated. (D) DC or (E) PMNs were cultured alone or with hepatic γδT cells and tested for expression of respective activation markers. Differences in activation marker expression were determined by calculating relative MFIs between groups in experiments repeated 3 times (*p<0.05, **p<0.01, ***p<0.001).

FIG. 28. γδT cell-derived IL-17 promotes liver regeneration. (A) γδT cells from the livers of regenerating WT and CD45.1 mice were tested for IL-17 expression. (B) Liver from regenerating WT and CD45.1 mice were tested for co-expression of IL-17 and CD45.2 or CD45.1, respectively. (C-E) WT and CD45.1 mice underwent partial hepatectomy and were tested at 36 h for hepatocyte expression of (C) PCNA by IHC, (D) Cyclin D1 by PCR, and (E) serum levels of IL-6 (n=3-5 mice/group). (F) WT mice underwent partial hepatectomy after administration of a neutralizing IL-17 mAb or isotype control and were tested at 36 h for hepatocyte expression of PCNA by IHC (n=4 mice/group; *p<0.05, **p<0.01, ***p<0.001).

FIG. 29. Dectin-1 and IL-22 promote liver regeneration. (A) Hepatocytes and CD45⁺ inflammatory cells from regenerating and sham-treated livers were gated and tested for expression of Dectin-1 ligands using an Fc(human):Dectin-1(mouse) fusion protein. These data suggest upregulation of Dectin-1 ligands in the regenerating liver. Representative data and averages of 3 replicate experiments are shown. (B) Data was confirmed by Western blotting using whole liver lysates. (C) WT, TCRδ^{-/-}, and TCRδ^{-/-} mice treated with recombinant IL-22 underwent hepatectomy and were tested for BrdU incorporation at 36 h (n=4/group). These data suggest that recombinant IL-22 rescues the rate of regeneration in TCRδ^{-/-} mice. (D, E) Liver regeneration was compared in WT and Dectin-1^{-/-} mice at 36 h by expression of (D) PCNA and (E) Cyclin D1 suggesting a retarded rate of regeneration in the context of Dectin-1 deletion (n=5 mice/group; *p<0.05, **p<0.01, ***p<0.101).

### DESCRIPTION OF THE DISCLOSURE

The present disclosure is based on the identification of the role of Dectin-1 in liver function and disorders, and provides methods for treatment of liver disorders involving modulation of Dectin-1 signaling pathway. The disclosure further provides methods for identifying Dectin-1 pathway activators for the treatment of liver disorders.

Data presented herein demonstrates that Dectin-1 suppresses TLR4 activation and provides an example of negative regulation between PRRs in an in vivo model of sterile inflammation or LPS-mediated sepsis. Dectin-1^{-/-} mice were found to have increased inflammatory responses, toxicity, and mortality from LPS-induced sepsis, and Dectin-1^{-/-} leukocytes and HSC exhibit higher activation states in response to TLR4 ligation. Exacerbated hepatic fibrosis in Dectin-1^{-/-} liver, and augmented LPS-induced sepsis in Dectin-1^{-/-} animals, is associated with elevated expression of TLR4 and its co-receptor CD14 in innate inflammatory cells. This finding is particularly notable as expression levels of TLR4 and CD14 are similar in WT and Dectin-1^{-/-} leukocytes at baseline; however, in liver fibrosis, the upsurge in TLR4 and CD14 expression—which we found to be characteristic of toxin-induced hepatic injury—is reduced in Dectin-1-expressing leukocytes. CD14 blockade mitigated exacerbated hepatic fibrosis and LPS-induced sepsis in Dectin-1^{-/-} mice but had negligible effects on liver fibrosis or systemic inflammation in WT animals. Given the central position of CD14 in TLR4 mediated inflammatory responses, this is surprising and suggests that CD14 is dispensable for both intra- and extra-hepatic TLR4-mediated inflammatory responses. Below a certain threshold CD14 level, CD14 expression may be dispensable in TLR4-mediated inflammation whereas above this threshold level, CD14 blockade suppresses TLR4 responses. Using animal models for LPS-induced endotoxemia and liver fibro-inflammation, we also demonstrate that Dectin-1 ligation in vivo protected animals from liver disorders.

In one embodiment, the disclosure provides a method for treating liver disorders including one or more of the following: hepatocellular carcinoma, liver fibrosis, liver cirrhosis, sterile inflammation, and LPS induced liver inflammation. The method comprises administering to an individual a therapeutically effective amount of one or more activators of Dectin-1 pathway. An activator of Dectin-1 pathway may be an agonist of Dectin-1 (such as a ligand that binds to Dectin-1) or may activate (or suppress) a downstream event similar to the action of Dectin-1. Downstream events in the Dectin-1 pathway following Dectin-1 ligation include suppression of TLR4 activation and suppression and CD14 expression. TLR suppression can be measured as reduced expression of TLR4.

The term “Dectin-1 agonist”, also referred to herein as “Dectin-1 ligand”, refers to a molecule that specifically binds to Dectin-1 resulting in activation of the Dectin-1 pathway.

The term “therapeutically effective amount” as used herein refers to an amount of an agent sufficient to achieve, in a single or multiple doses, the intended purpose of treatment. For example, an effective amount to treat HCC is an amount sufficient to kill HCC cells. The exact amount desired or required will vary depending on the particular compound or composition used, its mode of administration and the like. Appropriate effective amount can be determined by one of ordinary skill in the art informed by the instant disclosure using only routine experimentation.

Within the meaning of the disclosure, “treatment” also includes relapse, or prophylaxis as well as the treatment of acute or chronic signs, symptoms and/or malfunctions. The treatment can be orientated symptomatically, for example, to suppress symptoms. It can be effected over a short period, over a medium term, or can be a long-term treatment, for example within the context of a maintenance therapy.

The pharmaceutical composition of the invention may be administered in any route that is appropriate, including but not limited to parenteral or oral administration. The pharmaceutical compositions for parenteral administration include solutions, suspensions, emulsions, and solid injectable compositions that can be dissolved or suspended in a solvent immediately before use. The injections may be prepared by dissolving, suspending or emulsifying one or more of the active ingredients in a diluent. Examples of diluents are distilled water for injection, physiological saline, vegetable oil, alcohol, and combinations thereof. Further, injections may contain stabilizers, solubilizers, suspending agents, emulsifiers, soothing agents, buffers, preservatives, etc. The injections may be sterilized in the final formulation step or prepared by sterile procedure. The pharmaceutical compositions of the disclosure may be formulated into a sterile solid preparation, for example, by freeze-drying, and may be used after sterilized or dissolved in sterile injectable water or other sterile diluent(s) immediately before use. The compositions described can include one or more standard pharmaceutically acceptable carriers. Some examples herein of pharmaceutically acceptable carriers can be found in: Remington: The Science and Practice of Pharmacy (2005) 21st Edition, Philadelphia, Pa. Lippincott Williams & Wilkins.

Various methods known to those skilled in the art can be used to introduce (i.e., administer) the compositions of the disclosure to an individual. For example, an agent or mixture of agents, or compositions containing one or more active agents, can be administered in any manner including, but not limited to, orally, parenterally, sublingually, transdermally, rectally, transmucosally, topically, via inhalation, via buccal administration, or combinations thereof. Parenteral administration includes, but is not limited to, intravenous, intraarterial, intracranial, intradermal, subcutaneous, intraperitoneal, subcutaneous, intramuscular, intrathecal, and intraarticular. The agents(s) can also be administered in the form of an implant, which allows a slow release of the compound(s), as well as a slow controlled i.v. infusion.

When administered through oral route, the composition may be in a solid, semi-solid or liquid form. The solid compositions include tablets, pills, capsules, dispersible powders, granules, and the like. The capsules include hard capsules and soft capsules. In such solid compositions for oral use, one or more of the active compound(s) may be admixed solely or with diluents, binders, disintegrators, lubricants, stabilizers, solubilizers, and then formulated into a preparation in a conventional manner. The preparations may be slow-release preparations. Liquid compositions for oral administration include pharmaceutically acceptable aqueous solutions, suspensions, emulsions, syrups, elixirs, and the like. The compositions may also contain wetting agents, suspending agents, emulsifiers, sweetening agents, flavoring agents, preservatives, buffers and the like.

The individual to whom the present compositions are to be administered may be human or may be a non-human animal. For veterinary use, an agent or agents or pharmaceutically acceptable salts of such agents are administered as suitably acceptable formulation in accordance with normal veterinary practice. The veterinarian can readily determine the dosing regimen and route of administration that is most appropriate for a particular animal. Animals treatable by the present compounds and methods include pets, farm animals and the like.

The method of the present disclosure may be carried out in an individual who has been diagnosed with a liver disorder (i.e., therapeutic use) or may be carried out in an individual who is at risk of developing the disorder (i.e., prophylactic use). It may also be carried out in individuals who have a relapse or are at a risk of having a relapse after being treated for the liver disorder.

In one embodiment, the method comprises identifying an individual who is has a liver disorder and then administering a therapeutically effective amount of a Dectin-1 activator. In one embodiment, the method comprises identifying an individual who is at risk of developing a liver disorder and then administering a therapeutically effective amount of a Dectin-1 activator. The administration of Dectin-1 activator may be combined with other modalities of treatment such as including other chemotherapeutic or other therapeutic agents, surgery, radiation, immunotherapy and the like.

Activators of Dectin-1 signaling may be used alone, with other agents with similar or different effects or with other modalities, including chemotherapeutic agents, surgery, radiation and the like.

In one aspect, this disclosure provides a method for activating Dectin-1 signaling pathway. The method comprises contacting a cell, in which activation of Dectin-1 signaling pathway is desired, with an activator of Dectin-1 (such as an agonist). An agonist is also referred to herein as a ligand. In one embodiment, the cell is contacted with an agonistic antibody or a fragment thereof, or with a small molecule. Examples of Dectin-1 ligands include beta-glucan peptide (BGP), curdlan AL, heat-killed C. albicans, heat-killed S. cerevisiae, laminarin, lichenan, pustulan, schizophyllan, scleroglucan, WGP Dispersible, Zymosan, Zymosan Depleted. These agonists are commercially available (Invivogen). Another example of Dectin-1 activator is vimentin. Another example of Dectin-1 activator is an agonistic anti-Dectin-1 antibody, such as, for example, an antibody described in U.S. Pat. No. 9,045,542, the description of which antibody is incorporated herein by reference. In one embodiment, a cell is contacted with one or more of Dectin-1 agonists or activators.

In one aspect, this disclosure provides methods for identifying activators of Dectin-1 pathway in liver cells, such as liver leukocytes. In one embodiment, the activators of Dectin-1 pathway are Dectin-1 agonists. The activity of a test agent may be evaluated based on the effect on any step of the Dectin-1 pathway (as described in this disclosure). It can be compared to the effect in the absence of the test compound or may be compared to the effect of Dectin-1 or a known agonist thereof.

Assays to evaluate agents for binding to Dectin-1 may be carried out by in vitro using purified or recombinant Dectin-1. Assays can also be carried out in vitro using cells which express Dectin-1—such as liver leukocytes or hepatic stellate cells. Further, screening test may be carried out in vivo using animal models. The cells in culture may be primary cells or may be secondary cells or cell lines. Examples of suitable cells include liver leukocytes (such as dendritic cells, macrophages, CD14⁺ monocytic cells and the like), and hepatic stellate cells. The cells may be enriched from sources such as whole blood. For example, whole blood may be obtained from an individual and desired types of leukocytes may be isolated using well known techniques or using commercially available kits (such as kits from Miltenyi Biotec). In one embodiment, the cells may be modified cells. For example, the cells may be engineered to express or overexpress Dectin-1. The cells in culture can be maintained by using routine cell culture reagents and procedures. In one embodiment, the assays may be carried out in animals including mice after administration of Thioacetamide (TAA) or Carbon tetrachloride (CCl₄). In one embodiment, an LPS-induced sepsis model or a liver cancer model (DEN+CC14) may be used as animal models.

Various agents can also be tested for their effects on alleviating the symptoms or severity of liver disorders including liver cancer such as HCC, or liver fibrosis. The agents can also be tested on their effects on liver regeneration—which may be done using animals or may be done in culture using primary or secondary hepatocytes or cell lines.

The compounds for testing may be part of a library or may be newly synthesized. Further, the compounds may be purified, partially purified or may be present as cell extracts, crude mixtures and the like—i.e., unpurified. While it is ideal to test each compound separately, a combination of compounds may also be tested.

Test agents having a desired level of effect compared to a control may be selected from the screening tests described herein. In one embodiment, test agents are identified that have a statistically significant effect over a negative control. In one embodiment, test agents are identified that have at least 5% or 10% effect over a negative control. A negative control may be a sample in which an agent known not to have an effect is used or may be one that does not have the test agent. In one embodiment, a positive control that is known to have an effect may be used. For example, in one embodiment, the positive control is a Dectin-1 ligand (Lena). In one embodiment, test agents are identified that have at least the effect of Dectin-1 ligand. In one embodiment, test agents are identified that have up to 10%, 15%, 20%, or 25% less effect than a Dectin-1 ligand. These criteria and controls, both positive and negative, can be used for any of the tests described herein.

In other embodiments, candidate agents may be tested for effect on one or more steps involved in the Dectin-1 pathway such as events downstream of the Dectin-1 ligation. For example, events downstream of Dectin-1 ligation include a decrease in expression or function of TLR4, a decrease in expression or function of CD14, over-expression of signaling mechanism that down regulates NF-κB, or suppressed activation of NF-κB.

In one embodiment, Dectin-1 agonists may be identified by evaluating their effects on degradation of TLR-associated adaptor proteins, dissociation of TLR-dependent signaling complexes, and regulation of transcription of soluble inflammatory mediators.

In one embodiment, the disclosure provides pharmaceutical compositions comprising activators of Dectin-1 pathway. The pharmaceutical composition comprises one or more activators of Dectin-1 signaling and a pharmaceutically acceptable carrier.

The following examples further describe the disclosure. These examples are intended to be illustrative and not limiting in any way.

### Example 1

This example demonstrates that Dectin-1 regulates hepatic fibrosis and hepatocarcinogenesis by suppressing TLR signaling pathways.

Experimental Procedures

Animals and In Vivo Models of Liver Fibrosis, Hepatic Carcinogenesis, and Sepsis

C57BL/6 and CD45.1 mice were purchased from Jackson and bred in house. Dectin-1^{-/-} mice were a gift from Gordon Brown (University of Aberdeen). Mincle^{-/-} mice were obtained from the MMRRC. Age-matched 6-8 week old mice were used in all experiments. To induce hepatic fibrosis, female mice were treated with thrice weekly injections of TAA (250 mg/kg; Sigma) for 12 weeks as described (Connolly et al., 2009, The Journal of clinical investigation 119, 3213-3225). Alternatively, mice received bi-weekly injections of CCl₄ (0.5 ml/kg; Sigma) for the same duration. To induce HCC, male mice were injected i.p. at 2 weeks of age with a single dose of DEN (15 mg/kg, Sigma) followed by bi-weekly injections of CCl₄ (0.2 ml/kg) starting at 8 weeks of age ((Dapito et al., 2012, Cancer cell 21, 504-516). Mice were sacrificed 24 weeks later. To induce sepsis, male mice were injected i.p. with a single dose of LPS (15 mg/kg; Sigma). Rectal core body temperature was determined using a MicroTherma 2 temperature probe (Thermoworks). In selected experiments, MyD88 inhibitory peptide (1 mg/kg; Novus), an α-M-CSF neutralizing antibody (2 mg/kg; Clone 5A1, BioXCell), or an α-CD14 neutralizing antibody (4 mg/kg; Clone 4C1, BD Bioscience) was administered immediately preceding each TAA or LPS administration. At the time of sacrifice, blood and liver samples were harvested for analysis. Serum liver enzymes, including alanine aminotransferase (ALT) and aspartate aminotransferase (AST), were determined using commercial kits (Sigma). All animal procedures were approved by the NYU School of Medicine IACUC.

Human and Murine Cellular Isolation and Culture

Murine hepatic NPC were collected as previously (Connolly et al., 2009, The Journal of clinical investigation 119, 3213-3225). Briefly, the portal vein was cannulated and infused with 1% Collagenase IV (Sigma). The liver was then removed, minced, and filtered to obtain single cell suspensions. Hepatocytes were excluded with serial low speed (400 RPM) centrifugation followed by high speed (2000 RPM) centrifugation to isolate the NPC, which were then further enriched over a 40% Optiprep (Sigma) gradient. Human liver NPCs were isolated using a similar protocol as we have described ((Ibrahim et al., 2012, Gastroenterology 143, 1061-1072). Human PBMC were isolated by overlaying whole blood diluted 1:1 in PBS over an equal amount of Ficoll. The cells were then spun at 2100 RPM for 21 min at 20° C. and buffy coat harvested to obtain the PBMC as described ((Rehman et al., 2013, Journal of immunology 190, 4640-4649). Single-cell suspensions of murine splenocytes were isolated by manual disruption of whole spleen and RBC lysis. For HSC isolation, the liver was perfused with 1% Collagenase IV and HSCs were enriched over a 2-layer Nicodenz (Sigma) gradient. HSC were used for experiments on day 14 of culture. For cytokine analysis, HSC were cultured in complete medium (DNEM F12 with 10% heat-inactivated FBS, 2 mM L-glutamine, 100 U/ml penicillin and 100 g/ml streptomycin) at a concentration of 1×10⁶ cells/ml for 24 hours before supernatant harvest and analysis using a cytometric bead array according the manufactures' protocol (BD Biosciences). In vitro proliferation was measured using the XTT cell proliferation kit (Roche). PKC activity was measured using a PKC Kinase Activity Assay Kit (Abcam). In selected experiments, cellular activation was accomplished using LPS-EB Ultrapure (10 mg/ml), HKLM (108 cell/ml), HKCA (108 cells/ml), Zymosan Depleted (100 mg/ml), WGP Dispersible (100 mg/ml), Curdlan AL (100 mg/ml; all Invivogen), or recombinant murine M-CSF (100 ng/ml; R&D). In select experiments, cells were treated with mAbs directed against CD14 (10 mg/ml; 4C1), TNF-a (5 mg/ml; 2E2; Sloan-Kettering Institute), or a selective inhibitor of Protein Kinase C (GF109203X, 1 mM; Tocris Bioscience).

Flow Cytometry

Single-cell suspensions of liver or spleen cells or cultured HSC were incubated with Fc blocking reagent (Biolegend) for 10 min followed by 30 min incubation with fluorescently-conjugated mAbs directed against mouse CD11b (M1/70), CD11c (N418), CD45 (30-F11), F/480 (BM8), Gr1 (RB6-8C5), CD115 (AFS98), and MHC II (M5/114.15.2; all Biolegend). Cells were also tested for expression of Dectin-1 (2A11; Abcam), TLR4 (SA15-21; Biolegend) and CD14 (Sa14-2; Biolegend). Human liver NPC and PBMC were stained with mAbs directed against CD45 (HI30), CD14 (M5E2; both Biolegend), or Dectin-1 (259931; R&D). For intracellular cytokine staining, liver NPC were incubated for 4 hours with Brefeldin A (1:1000) before permeabilization of cells and staining using fluorescent conjugated mAbs against murine TNF-α (MP6-XT22;) or IL-6 (MP5-20F3; both Biolegend). Experiments were performed using the LSRII cytometer (BD Biosciences) and analysis was done using FlowJo software version 9.2 (Tree Star).

Histology, Immunohistochemistry and Immunofluorescence

Liver tissues were fixed overnight in 10% formaldehyde and were embedded in paraffin. Slides were stained with H&E, Masson Trichrome, or picric acid-Sirius red. For immunohistochemical analysis, sections were incubated with antibodies against mouse CD45 (30-F11; BD Bioscience), CD68 (KP1; Abcam), MPO (Rabbit polyclonal; Abcam), Dectin-1 (R1-8g7; Invivogen), PCNA (PC10; Biolegend), TLR4 (Rabbit polyclonal; Abcam), α-SMA (1A4; Abcam), Phalloidin (Alexa Flour 647, Cell Signaling), or M-CSF (Rabbit polyclonal; Abcam). Human liver sections were stained with an antibody against Dectin-1 (Rabbit polyclonal; Abcam). Quantification was performed by examining 10 high powered fields (HPFs) per slide. Fibrosis was quantified based on Trichrome staining using a computerized grid as described (Ochi et al., 2012, The Journal of clinical investigation 122, 4118-4129). Immunofluorescent imaging was performed using a LSM 700 confocal microscope and an Axiovert camera (Zeiss).

Western Blotting and Immunoprecipitation

For Western blotting, total protein was isolated from 75 mg liver tissue by homogenization in RIPA buffer with Complete Protease Inhibitor cocktail (Roche). Proteins were separated from larger fragments by centrifugation at 14000×g. After determining total protein by the Bradford protein assay, 10% polyacrylamide gels (NuPage, Invitrogen) were equiloaded, electrophoresed at 200 V, electrotransferred to PVDF membranes, and probed with monoclonal antibodies to b-actin, MMP2, MMP7, MMP9, TIMP2, TIMP7, p-Erk1/2, Erk1/2, p-NF-kb, NF-kb, MyD88, TRAF6, TRIF, and pPDGFRa (all Cell Signaling Technology) using the manufacturer's recommended concentrations. Blots were developed by ECL (Thermo Scientific). For immunoprecipitation experiments, TLR4 or Dectin-1 was precipitated with protein G-agarose. Immuno-precipitates were re-suspended and heated in loading buffer under reduced condition and resolved by 10% SDS-PAGE before transfer to PVDF membranes. The presence of the co-immunoprecipitated proteins were determined by western blotting.

mRNA Analysis

For PCR analysis, total RNA was extracted using the RNEasy Mini Kit (Qiagen) and cDNA made using the High Capacity Reverse Transcription kit (Applied Biosystems). RT-PCR was performed for mouse CD14 and β-Actin using commercially available pre-designed primers (Qiagen). For the mouse Fibrosis PCR array, Oncogene PCR array, and Cytokine and Chemokine PCR array, mRNAs were reverse transcribed into first-strand cDNA using an RT²miRNA First-Strand Kit (all Qiagen). RT² SYBR Green/ROX Quantitative PCR Master Mix (Qiagen) was used for amplification and the samples were run on the Stratagene Mx3005P. For Nanostring analysis, the nCounter mouse inflammation panel was employed using the nCounter Analysis System (Nanostring).

Statistical Analysis

Data is presented as mean+/−standard error of mean. Survival was measured according to the Kaplan-Meier method. Statistical significance was determined by the Student's t test and the log-rank test using GraphPad Prism 6 (GraphPad Software). P-values of <0.05 were considered significant.

Results

Dectin-1 Expression is Increased in Liver Fibrosis

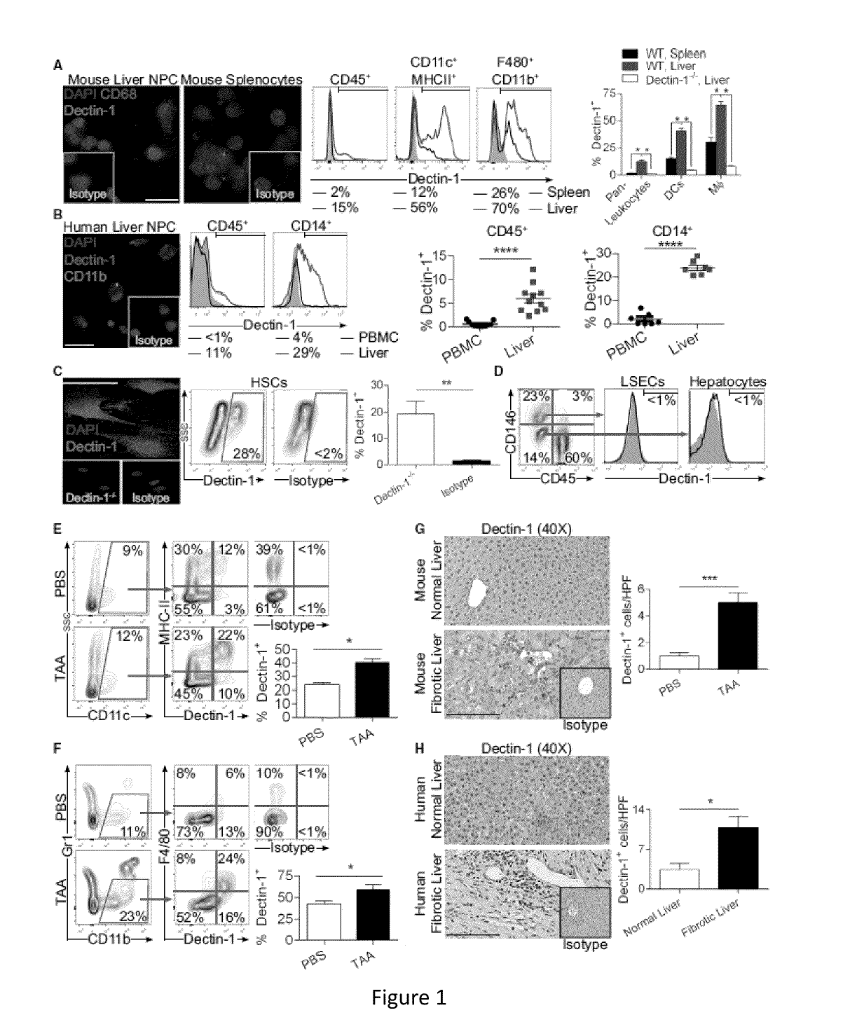

To assess the potential impact of Dectin-1 signaling in liver disease, we examined Dectin-1 expression in both hepatic inflammatory and parenchymal cells. Liver leukocytes, specifically dendritic cells (DCs) and macrophages, expressed high Dectin-1 compared with their counterparts in the murine spleen (FIG. 1A). Similarly, human liver leukocytes, and in particular CD14+ monocytic cells, expressed elevated Dectin-1 compared with peripheral blood mononuclear cells (PBMCs) (FIG. 1B). Hepatic stellate cells also expressed high Dectin-1 on immunofluorescence microscopy and flow cytometry (FIG. 1C). Conversely, liver parenchymal cells, including hepatocytes and liver sinusoidal endothelial cells, expressed minimal Dectin-1 (FIG. 1D). Moreover, in hepatic fibrosis, Dectin-1 expression was upregulated in hepatic DC (FIG. 1E) and macrophages (FIG. 1F) compared with their expression in normal liver. Accordingly, both murine (FIG. 1G) and human (FIG. 1H) liver fibrosis were associated with a robust intra-hepatic influx of Dectin-1+ cells.

Dectin-1 Regulates Hepatic Fibrosis

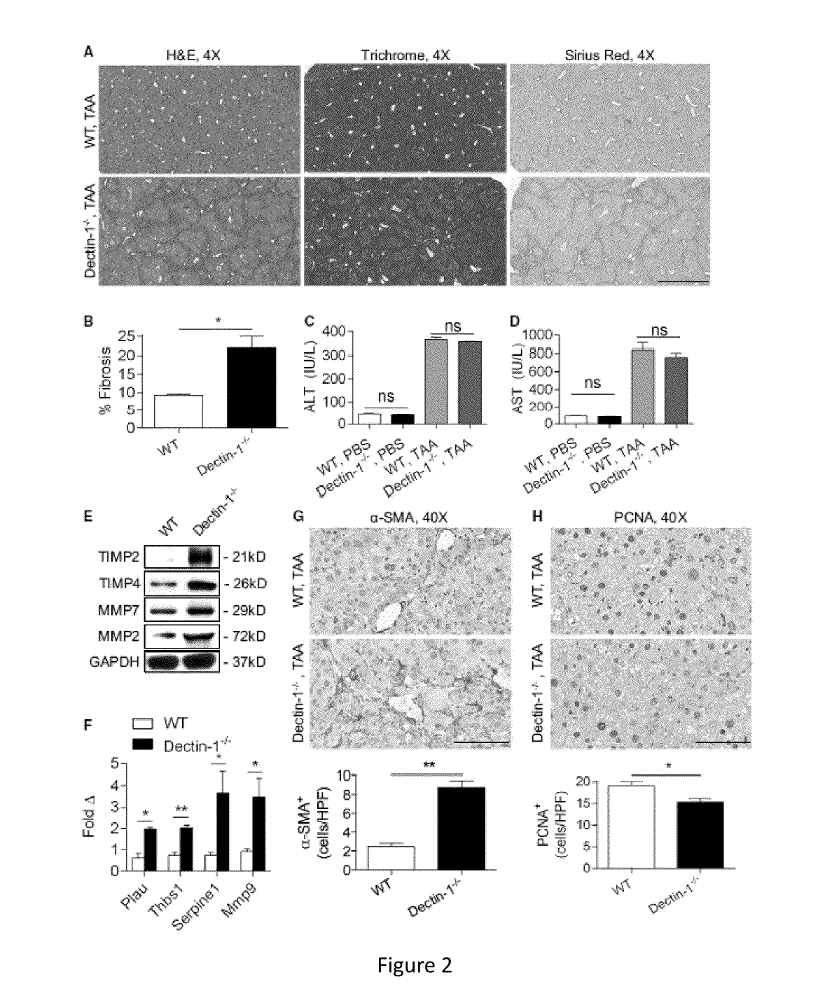

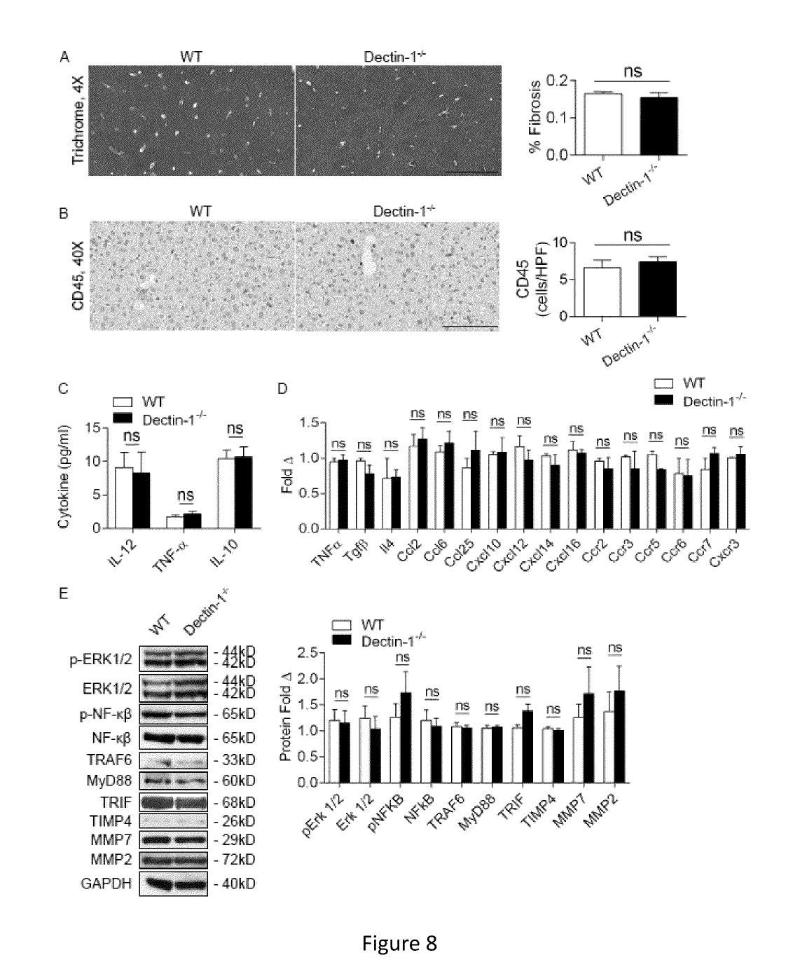

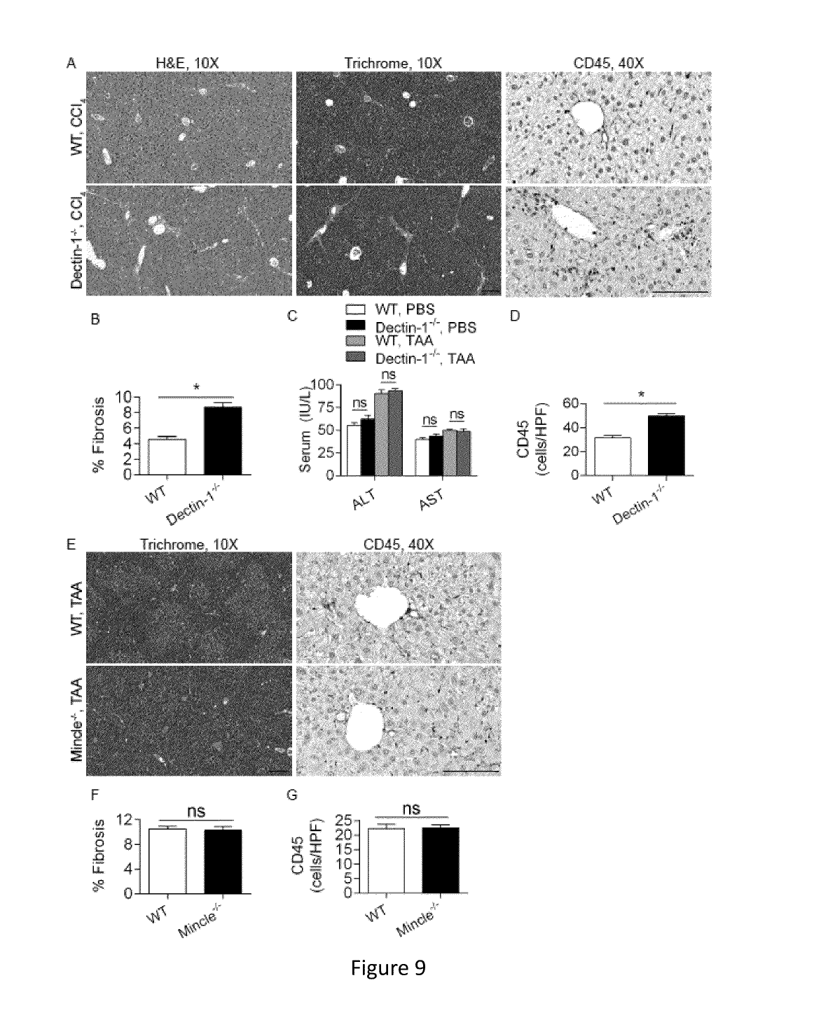

To test the importance of Dectin-1 signaling in modulating chronic liver disease, we induced hepatic fibrosis in wild-type (WT) or Dectin-1−/− mice using Thioacetamide (TAA) or Carbon tetrachloride (CC14). Non-fibrotic WT and Dectin-1−/− livers exhibited indistinguishable hepatic phenotypes (FIG. 8). However, Dectin-1-deficient livers developed markedly exaggerated hepatic fibrosis on histologic analysis in both the TAA (FIGS. 2A and 2B) and CC14 (FIGS. 9A-9D) disease models. Serum transaminase levels were not differentially elevated in Dectin-1−/− animals (FIGS. 2C, 2D, and 9C). Western blotting confirmed higher hepatic expression of extracellular matrix (ECM) proteins TIMP2, TIMP4, MMP7, and MMP2 in TAA-treated Dectin-1−/− liver compared with WT (FIG. 2E). Genes that regulate ECM remodeling were also expressed at higher levels in fibrotic Dectin-1−/− liver on RT-PCR analysis (FIG. 2F). Accordingly, a-SMA expression was elevated in fibrotic Dectin-1−/− liver, indicative of enhanced HSC activation (FIG. 2G). Furthermore, consistent with more advanced disease, there was diminished hepatocyte proliferation in the regenerative nodules of fibrotic Dectin-1−/− liver compared with WT (FIG. 2H), which is consistent with more advanced disease. Notably, mice deficient in Mincle—a C-type lectin receptor akin to Dectin-1—did not exhibit exacerbated hepatic fibro-inflammatory disease after hepatotoxin treatment suggesting specificity of the effect to Dectin-1 (FIGS. 9E-9G).

Dectin-1 Regulates Intra-Hepatic Inflammation

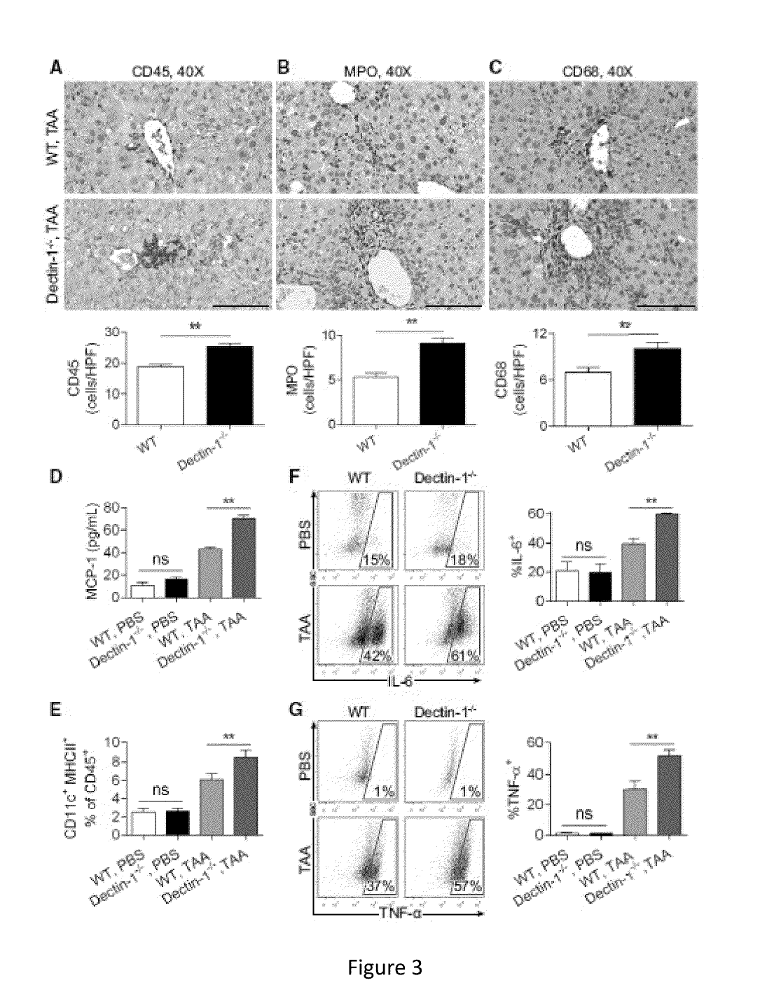

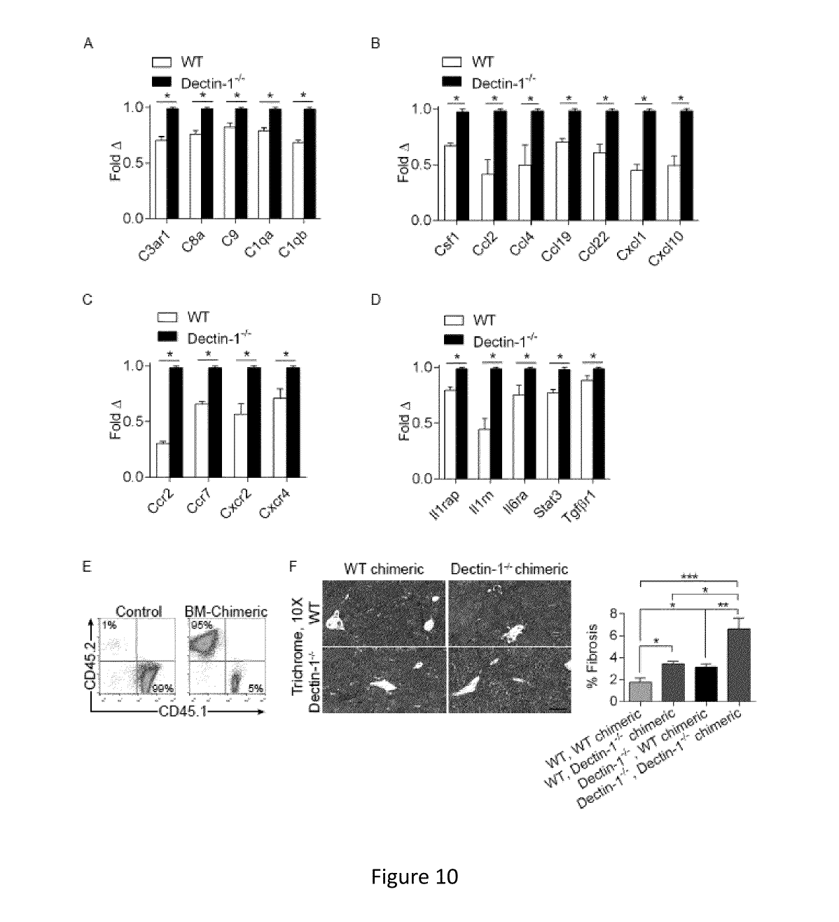

To test whether Dectin-1 suppresses hepatic inflammation in liver fibrosis, we analyzed the comparative innate immune infiltrates in TAA- or CC14-treated WT and Dectin-1−/− liver. Fibrotic Dectin-1−/− liver exhibited a higher pan-leukocyte infiltrate (FIGS. 3A, 9A and 9D), greater neutrophilia (FIG. 3B), and a higher influx of CD68+ macrophages (FIG. 3C) compared with WT. Consistent with these findings, serum levels of MCP-1 were elevated in fibrotic Dectin-1−/− liver (FIG. 3D). The fraction of DC was increased in the liver of TAA-treated Dectin-1−/− mice compared with WT (FIG. 3E). Further, intracellular cytokine analysis showed markedly higher interleukin 6 (IL-6) (FIG. 3F) and TNF-a (FIG. 3G) expression in APC in fibrotic Dectin-1−/− liver compared with WT. Collectively, these data indicate that deletion of Dectin-1 results in an expanded and more activated innate inflammatory infiltrate in hepatic fibrosis. To further assess the effect of Dectin-1 deletion on intra-hepatic inflammation in liver fibrosis, we tested hepatic expression of inflammatory mediators by nanostring analysis. We found higher expression of multiple components of the complement system in fibrotic Dectin-1−/− liver compared with WT (FIG. 10A). Further, Dectin-1 deletion in liver fibrosis resulted in exaggerated expression of pro-inflammatory chemokines (FIG. 10B), a diverse array of chemokine receptors (FIG. 10C), and genes associated with IL-1, IL-6, and TGF-b signaling (FIG. 10D). Notably, expression levels of soluble inflammatory mediators and chemokine receptors were not significantly different between PBS-treated WT and Dectin-1−/− liver (FIG. 8D). To determine in which compartment Dectin-1 signaling modulated liver fibrosis, we irradiated WT and Dectin-1−/− mice and made these animals chimeric using bone marrow derived from WT and Dectin-1−/− donors before inducing TAA-mediated liver fibrosis 7 weeks later. Our extent of chimerism was 95% (FIG. 10E). We found that Dectin-1 deletion in both the radio-sensitive and radio-resistant compartments had additive effects on exacerbating fibrosis (FIG. 10F).

Dectin-1 Protects Against Hepatocellular Carcinogenesis

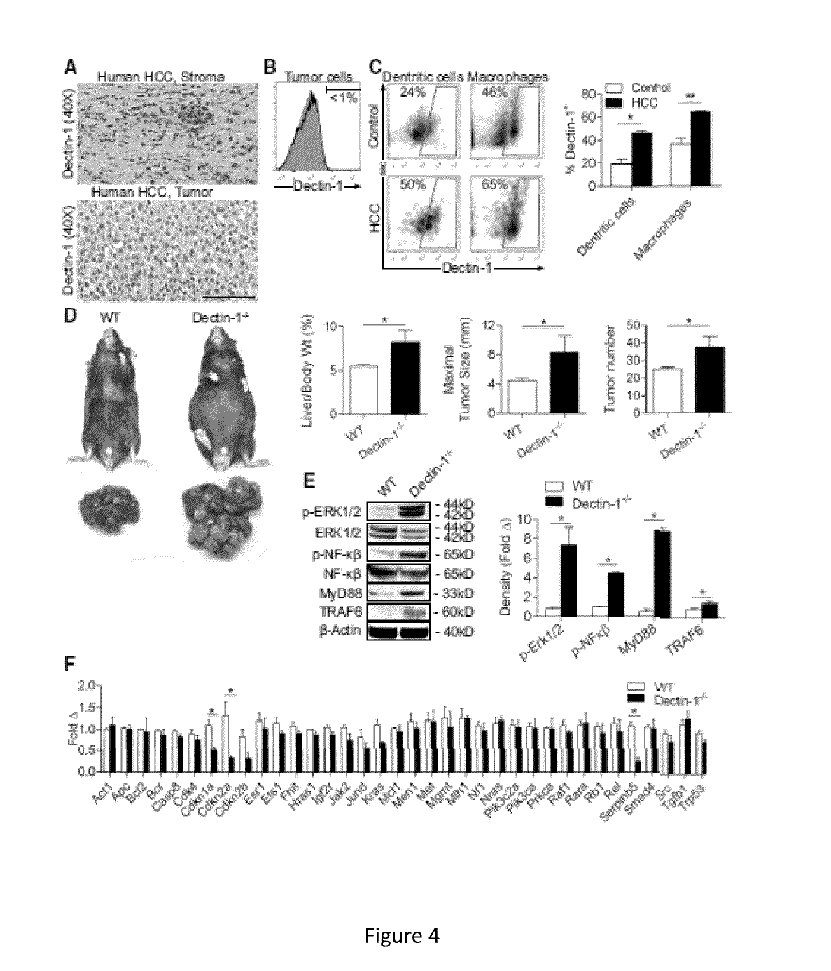

Hepatocellular carcinoma (HCC) develops almost exclusively in the setting of chronic liver disease. We found that HCC in humans is associated with a robust infiltrate of Dectin-1+ leukocytes (FIG. 4A). Tumor cells did not express Dectin-1 (FIGS. 4A and 4B). However, murine DC and macrophages upregulated their expression of Dectin-1 in HCC nodules compared with normal liver (FIG. 4C). Therefore, we postulated that, besides modulating liver fibrosis, Dectin-1 may also regulate hepatocellular oncogenesis. Mice were induced to develop HCC using diethylnitrosamine (DEN) and CC14. WT and Dectin-1−/− animals did not exhibit differential acute responses to DEN (data not shown). However, Dectin-1−/− mice developed accelerated hepatocellular carcinogenesis based on liver weight, tumor size, and number of nodules (FIG. 4D). Western blotting revealed evidence of higher MAP kinase and NF-kB signaling in Dectin-1−/− HCC tumor nodules, consistent with an aggressive malignant phenotype (FIG. 4E), whereas inflammatory signaling was similar in PBS-treated WT and Dectin-1−/− liver (FIG. 8E). MyD88 and TRAF6 expression were also elevated in Dectin-1−/− tumor nodules (FIG. 4E). Further, PCR array analysis showed that Cdkn1, Cdkn2, and Serpinb5—whose reduced expression is associated with a more aggressive HCC phenotype—were each expressed at lower levels in Dectin-1−/− tumors compared with WT (FIG. 4F).

Dectin-1 Suppresses TLR4 Activation

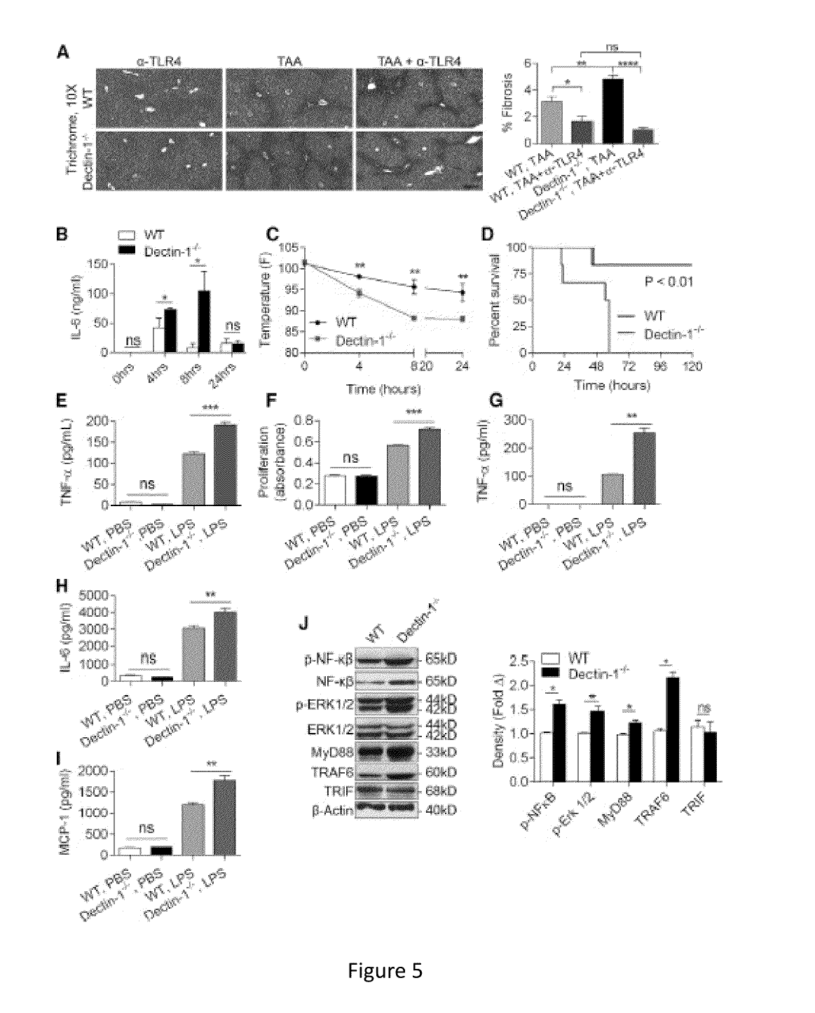

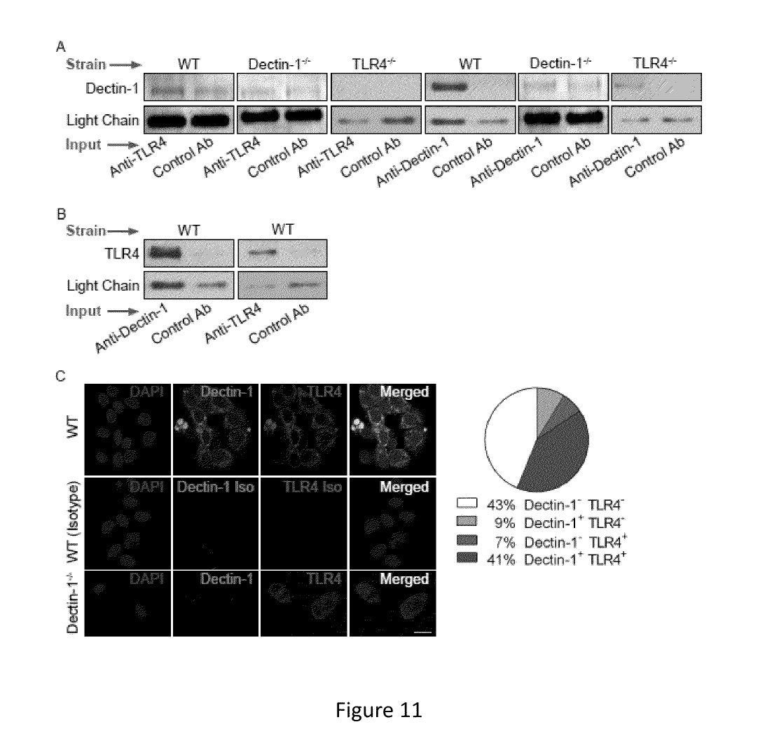

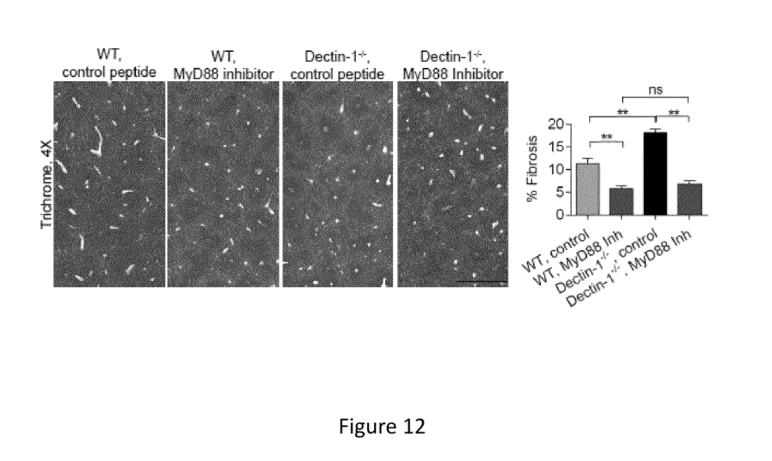

Dectin-1 has not previously been linked to sterile inflammation or oncogenesis. We found that TLR4 and Dectin-1 co-associate in liver inflammatory cells as evidenced by immunoprecipitation experiments (FIGS. 11A and 11B) and confocal microscopy (FIG. 11C) suggesting opportunity for cross-regulation. Therefore, we investigated if suppression of TLR4 signaling may be the mechanism of Dectin-1-mediated protection in chronic liver disease. The data presented herein shows that inhibition of MyD88 (FIG. 12A) or TLR4 (FIG. 5A) abrogated the exacerbated fibrosis associated with Dectin-1 deletion. To directly test whether Dectin-1 suppresses TLR4 signaling, we induced endotoxemia by challenging WT and Dectin-1−/− mice with TLR4 ligand LPS. Serum IL-6 levels were substantially higher and more sustained in Dectin-1−/− mice compared with WT (FIG. 5B). Dectin-1−/− mice also exhibited a sharper decrease in core temperature after LPS challenge suggesting increased systemic toxicity resulting from TLR4 ligation (FIG. 5C). Moreover, Kaplan-Meier analysis revealed markedly diminished survival in LPS-treated Dectin-1−/− mice compared to WT (FIG. 5D). Taken together, these data suggest that Dectin-1 inhibits TLR4 activation in vivo. Similarly, in vitro LPS-treatment of splenocytes from Dectin-1−/− mice resulted in higher TNF-a production (FIG. 5E) and proliferation (FIG. 5F) compared with WT. Splenocyte composition was similar in WT and Dectin-1−/− animals (data not shown). LPS treatment of Dectin-1−/− HSCs also resulted in higher TNF-a (FIG. 5G), IL-6 (FIG. 5H), and MCP-1 (FIG. 5I) production compared with LPS-treated WT HSCs. Notably, Dectin-1 deletion did not result in upregulated cytokine responses to TLR2 (HKLM) ligation (data not shown).

To test for evidence of Dectin-1 suppression of TLR4 signaling in hepatic fibrosis, tissues from fibrotic WT and Dectin-1−/− liver were probed for expression of TLR4-related signaling intermediates. We found elevated expression of TRAF6, MyD88, and activated NF-kB and MAP Kinase signaling intermediates in fibrotic Dectin-1−/− liver compared with WT (FIG. 5J), consistent with higher TLR4 activation. Conversely, the TRIF adaptor protein was expressed at similar levels in both groups (FIG. 5J).

Dectin-1 Critically Regulates Expression of TLR4 and CD14

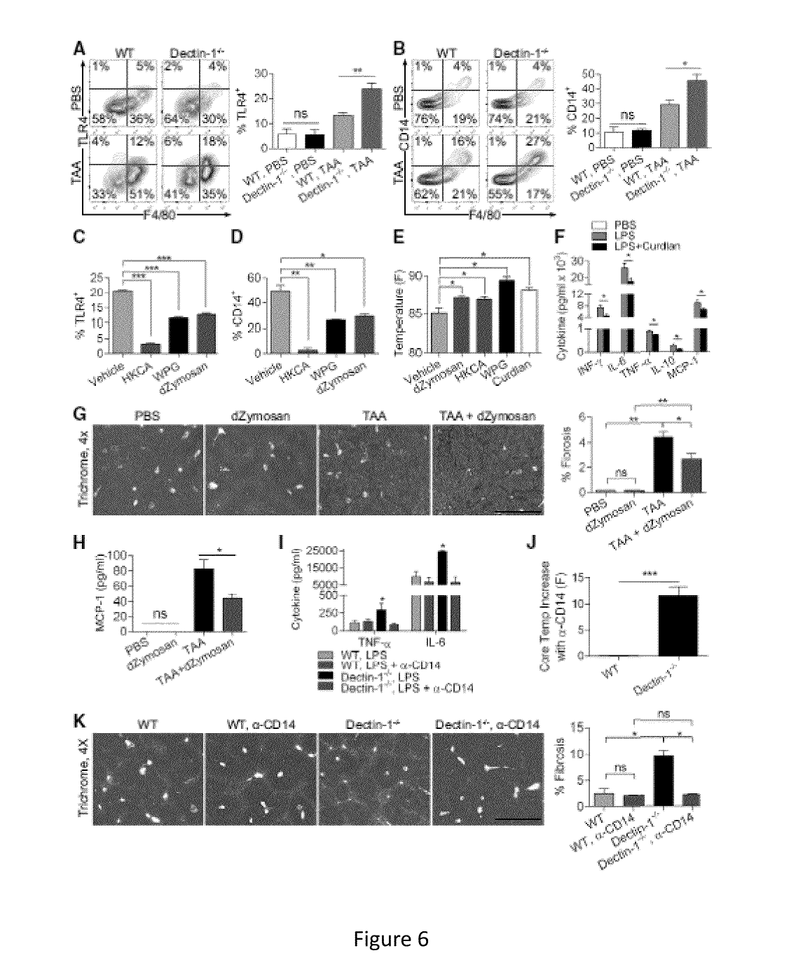

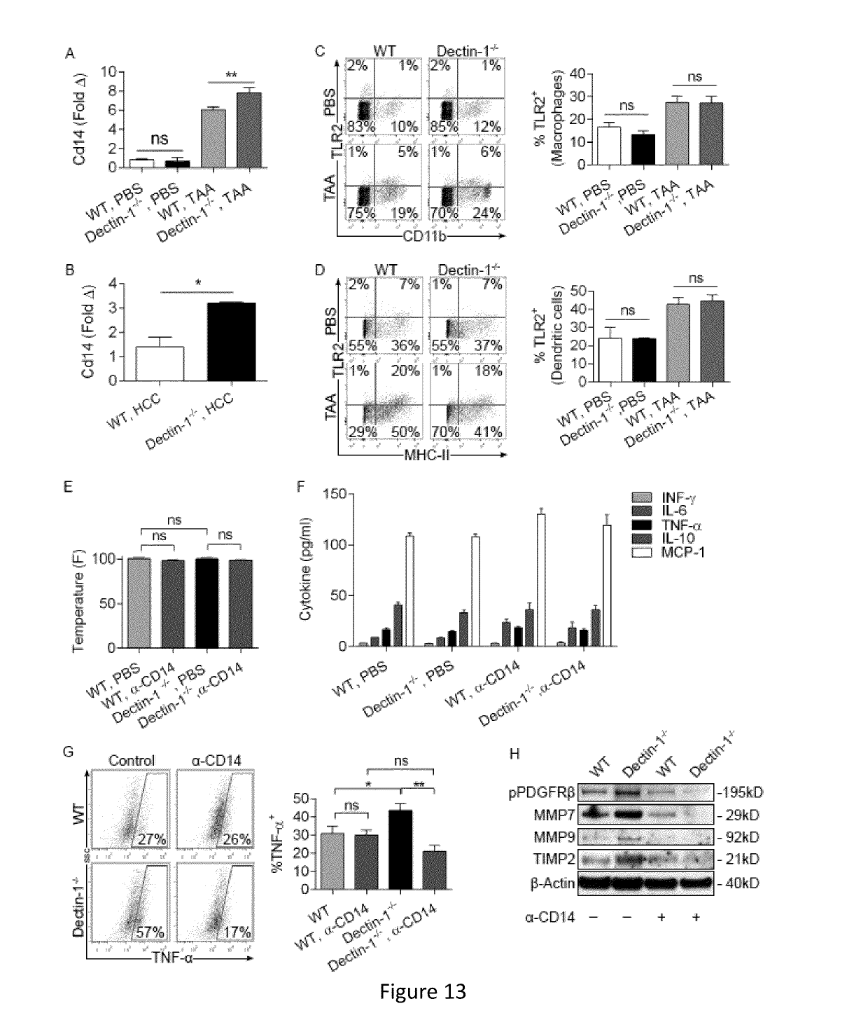

We investigated if Dectin-1 modulated TLR4 activation by suppressing TLR4 expression. We observed that whereas TLR4 was expressed at similar levels in PBS-treated WT and Dectin-1−/− hepatic APC, in liver fibrosis TLR4 was differentially upregulated in Dectin-1−/− macrophages (FIG. 6A) compared with WT. Further, CD14—a critical co-receptor for TLR4—was also expressed at elevated levels in macrophages derived from fibrotic Dectin-1−/− liver (FIG. 6B). Similar upregulations in TLR4 and CD14 expression were seen in Dectin-1−/− DC (data not shown) and on PCR of fibrotic whole liver specimens (FIG. 13A). Analysis of murine HCC tumors also revealed elevated Cd14 expression in Dectin-1−/− tumor nodules compared with WT (FIG. 13B). By contrast, Dectin-1 ligation using a variety of ligands substantially lowered macrophage expression of TLR4 (FIG. 6C) and CD14 (FIG. 6D). Further, Dectin-1 ligation in vivo protected animals from LPS-induced endotoxemia (FIGS. 6E and 6F) and liver fibro-inflammation (FIGS. 6G and 6H). Notably, Dectin-1−/− macrophages or DC did not exhibit elevated expression of TLR2 in hepatic fibrosis (FIGS. 13C and 13D) or after stimulation with TLR2 ligand HKLM compared with WT (data not shown).

To test whether Dectin-1 suppression of CD14 expression is a primary mechanism in the capacity of Dectin-1 to mitigate TLR4 responsiveness, we blocked CD14 in vivo coincident with PBS or LPS challenge in WT and Dectin-1−/− mice. CD14 blockade had no discernible effect in PBS-treated WT or Dectin-1−/− mice (FIGS. 13E and 13F). However, blockade of CD14 abrogated the augmented LPS-mediated inflammatory responses (FIG. 61) and systemic toxicity (FIG. 6J) exclusively in Dectin-1−/− animals. Moreover, CD14 blockade was markedly protective against hepatic fibrosis in TAA-treated Dectin-1−/− liver, whereas CD14 inhibition had imperceptible effects in WT liver (FIG. 6K). Similarly, CD14 inhibition reduced APC activation in fibrotic Dectin-1−/− liver but did not have anti-inflammatory effects in fibrotic WT liver (FIG. 13G). In our parallel in vitro experiments, CD14 blockade was also more inhibitory in LPS-stimulated Dectin-1−/− HSCs compared with WT HSCs in terms of cellular activation (pPDGFR) and ECM protein production (MMP7, MMP9, TIMP2) (FIG. 13H). Collectively, these data imply that Dectin-1 regulates TLR4 signaling in LPS sepsis as well as in liver fibrosis by modulating CD14 levels.

M-CSF Promotes CD14 Expression in Dectin-1−/− Liver

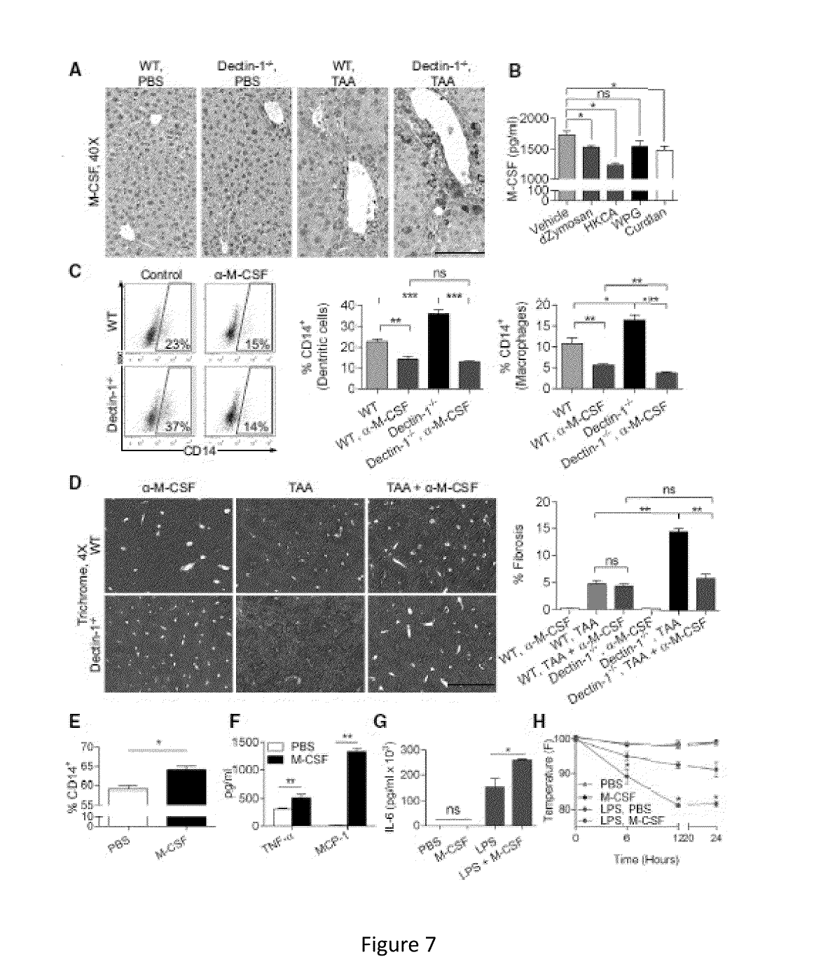

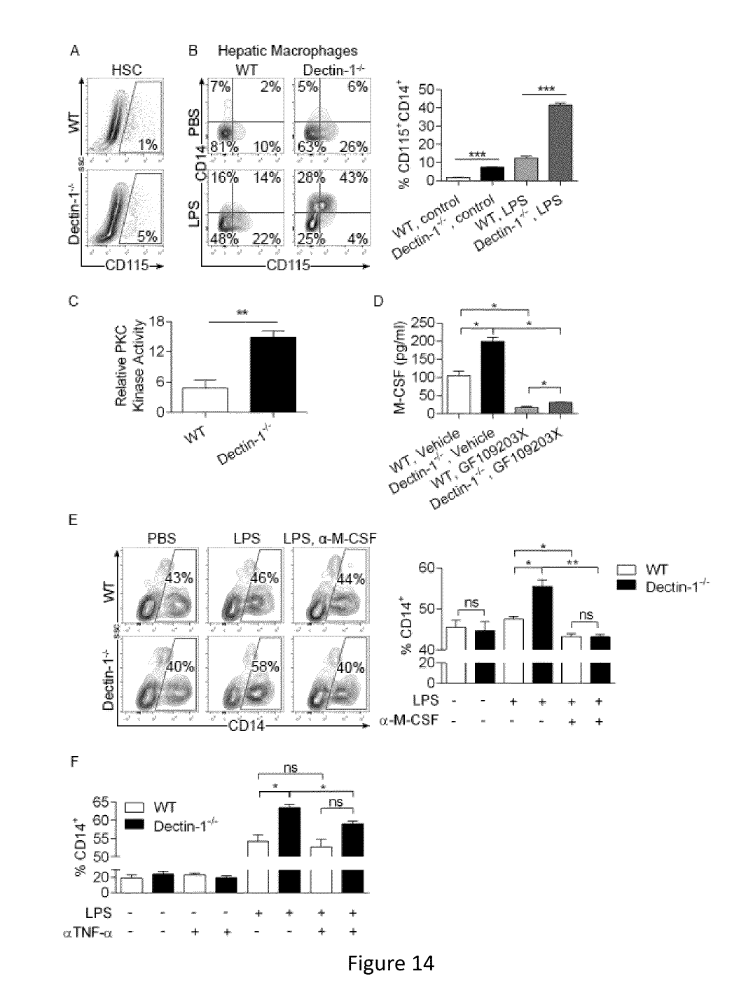

We discovered that deletion of Dectin-1 in the fibrotic liver increased expression of M-CSF in hepatic inflammatory and parenchymal cells based on immunohistochemical (FIG. 7A) and mRNA (FIG. 10B) analyses. Dectin-1 deletion also upregulated M-CSF receptor CD115 expression in HSC cultures (FIG. 14A) and in liver APC (FIG. 14B). By contrast, Dectin-1 ligation lowered M-CSF expression in vivo after LPS treatment (FIG. 7B). We found that Protein Kinase C (PKC)—which can regulate M-CSF activity—was upregulated in the context of Dectin-1 deletion (FIG. 14C) and PKC inhibition abrogated the higher M-CSF expression (FIG. 14D). We investigated if augmented M-CSF signaling is responsible for the pathologically high CD14 expression and the exacerbated hepatic fibrosis in Dectin-1−/− liver. We observed that in vivo M-CSF blockade during fibrogenesis resulted in markedly lower CD14 expression in Dectin-1−/− hepatic APC with smaller effects in WT (FIG. 7C). Similarly, in vitro M-CSF blockade mitigated the higher CD14 expression in LPS-stimulated Dectin-1−/− macrophages but had negligible effects in WT (FIG. 14E). Moreover, similar to CD14 blockade, M-CSF inhibition ameliorated hepatic fibrosis in the setting of Dectin-1 deletion but offered no protection in WT liver (FIG. 7D). Conversely, treatment with recombinant M-CSF increased CD14 expression (FIG. 7E) and upregulated MCP-1 and TNF-a expression in macrophages in vitro (FIG. 7F) and exacerbated LPS-mediated sepsis in vivo (FIGS. 7G and 7H). TNF-a blockade prevented the M-CSF-induced differential CD14 upregulation in Dectin-1−/− macrophages (FIG. 14F). Collectively, these data support that Dectin-1-regulated expression of M-CSF drives CD14-dependent exaggerated hepatic fibrosis and inflammatory responses in LPS-mediated sepsis.

### Example 2

This example describes a role for Dectin-1 and its cognate ligands in hepatic regeneration, through their influence on the production of IL-17-family cytokines in hepatic γδT cells.

Materials & Methods

Animals and Model of Partial Hepatectomy

C57BL/6, TCRδ^{-/-}, and C57BL/6-Trdc^{tm1Mal} mice were purchased from Jackson (Bar Harbor, Me.). For selected experiments, CD45.1 mice and C57BL/6 controls were purchased from Taconic (Germantown, N.Y.). Dectin-1^{-/-} mice were a gift from Gordon Brown (University of Aberdeen). Bone marrow chimeric animals were created by irradiating mice (9 Gy) followed by i.v. bone marrow transfer (10⁷ cells) as described by us (Ochi et al., The Journal of clinical investigation 2012; 122:4118-29). The 70% partial hepatectomy procedure entailed ligation and removal of the left and median lobes of the liver in 8-12 week-old mice (Mitchell et al., Nat Protoc 2008; 3:1167-70). The sham operation consisted of a midline laparotomy alone. All animal procedures were approved by NYU School of Medicine IACUC.

Human and Murine Liver Cell Isolation

Murine hepatic non-parenchymal cells (NPC) were collected as previously described (Connolly et al., J Clin Invest 2009; 119:3213-25). Briefly, the portal vein was cannulated and infused with 1% Collagenase IV (Sigma, Saint Louis, Mo.). The liver was then removed, minced, and filtered to obtain single cell suspensions. Hepatocytes were excluded with serial low speed (300 RPM) centrifugation followed by high-speed (1500 RPM) centrifugation to isolate the NPC, which were then further enriched over a 40% Optiprep (Sigma) gradient (Connolly et al., J Clin Invest 2009; 119:3213-25). Human liver NPCs were isolated using a similar protocol as we have described (Ibrahim et al., Gastroenterology 2012; 143:1061-72). All studies on human tissue were carried out under an IRB-approved protocol.

Statistics

Data is presented as mean+standard error. Comparisons were made using student's t test for paired or unpaired samples. p<0.05 was considered significant.

Supplemental Materials & Methods

Modulation of Immunity in Liver Regeneration

In selected experiments, NK1.1⁺ cells were depleted using an mAb (PK136, 150 μg, eBiosciences, San Diego, Calif.). In some experiments weight-matched mice received a single i.p. dose of recombinant IL-22 (10 g, R&D, Minneapolis, Minn.) or an mAb directed against IL-17 (MM17F3, 150 μg, eBiosiciences, San Diego, Calif.) 1 h before hepatectomy. Alternatively, in selected experiments Zymosan depleted (Invivogen, San Diego, Calif.) was administered to mice i.p. in split doses of 0.25 mg/mouse immediately before and after hepatectomy.

Analysis of Liver Weight and Plasma and Serum Analysis

Gain in liver weight was determined at various time points after hepatectomy as described³. Plasma levels of Prothrombin were determined by ELISA (Abcam, Cambridge, Mass.). Serum Glucose levels were determined by using a glucometer (Nipro, Ft. Lauderdale, Fla.). AST and ALT were measured using a commercial kit (Sigma, St. Louis, Mo.).

Peripheral Blood Mononuclear Cell Isolation and Cell Culture

Human peripheral blood mononuclear cells (PBMC) were isolated by overlaying whole blood diluted 1:1 in PBS over an equal amount of Ficoll. The cells were then spun at 2100 RPM for 21 min at 20° C. and buffy coat harvested to obtain the PBMC. For murine in vitro cultures, cellular suspensions were plated in complete media (RPMI 1640 with 10% heat-inactivated FBS, 2 mM L-glutamine, 100 U/ml penicillin, 100 μg/mL streptomycin, and 0.05 mM 2-ME) at a density of 1×10⁶ cells/mL. In selected experiments, cells were stimulated with Zymosan Depleted (100 g/mL) alone or in the presence of an IL-17 mAb (8 ng/mL).

Flow Cytometry and Cytokine Analysis

Murine liver or spleen cells were incubated with Fc blocking reagent (Biolegend, San Diego, Calif.) for 10 minutes followed by incubation with mouse α-Galactosyl Ceramide-loaded CD1d Tetramers (ProImmune, Oxford, United Kingdom), or fluorescently-conjugated mAbs directed against mouse B220 (RA3-6B2), CCR6 (29-2L17), CD3e (145-2C11), CD4 (GK1.5), CD8 (53-6.7), CD11b (M1/70), CD11c (N418), CD19 (6D5), CD27 (LG.3A10), CD44 (IM7), CD45 (30-F11), CD54 (YN1/1.7.4), CD62L (MEL-14), CD68 (FA-11), CD69 (H1.2F3), CD86 (GL-1), FasL (MFL3), F/480 (BM8), Ly6G (1A8), MHC II (M5/114.15.2), NK1.1 (PK136), TCRγδ (UC7-13D5), TCRvγ1.1 (2.11), TCRvγ4 (UC3-10A6; all Biolegend), and Dectin-1 (2A11; Abcam, Cambridge, Mass.). Human liver NPC and PBMC were stained with mAbs directed against CD3 (HIT3a), CD45 (HI30), CD62L (DREG-56), CCR6 (G034E3), CD27 (0323), CD122 (TU27), TCRγδ (B1; all Biolegend), or Dectin-1 (R&D). For intracellular cytokine staining, freshly harvested liver NPC were incubated for 4-6 hours with Brefeldin A (1:1000), PMA (50 ng/mL), and Ionomycin (750 ng/mL) before permeabilization of cells and staining using fluorescent conjugated mAbs against murine IL-17 (TC11-18H10; BD) or IL-22 (Poly5164; Biolegend,) or anti-human IL-17 (BL168; Biolegend), IL-22 (22URTI; eBioscience). DAPI (Biolegend) was used for live cell discrimination after fixation and permeabilization. An Fc(human):Dectin-1(mouse) fusion protein (Enzo Lifesciences, Farmingdale, N.Y.) was used at a dose of 1 μg per million cells for flow cytometry. Experiments were performed using the LSRII (BD) and analysis was done using FlowJo software (Tree Star, Ashland, Oreg.). Serum cytokine levels were determined using a cytometric bead array (BD) or ELISA (IL-22; Biolegend) according to the respective manufacturer's protocol.

Immunohistochemistry

Liver tissues were fixed in formaldehyde, embedded in paraffin, and stained for B220 (RA3-6B2; BD), BrdU (Bu20a; Sigma), CCL20 (polyclonal, Abcam), CD45 (30-F11; BD), CD68 (KP1; Abcam), Ki67 (16A8; Biolegend), MPO (Rabbit polyclonal; LS Biosciences, Seattle, Wash.), PCNA (PC10; Biolegend). Liver leukocytes were also stained using mAbs against IL-17, IL-22, or Dectin-1. Immunofluorescent imaging was performed using an Axiovert 40 microscope (Zeiss, Thornwood, N.Y., USA). Fluorescent images were captured on an Axiovert 200M (Zeiss). For BrdU immunostaining, mice were injected i.p. with BrdU (100 g/g, Sigma) two hours before sacrifice. Data was quantified by examining 10 high-power fields (HPFs) per slide.

Western Blotting

For Western blotting, total protein was isolated from liver tissue by homogenization in RIPA buffer with Complete Protease Inhibitor cocktail (Roche, Pleasanton, Calif.). Proteins were separated from larger fragments by centrifugation at 14000×g. After determining total protein by the Bradford protein assay, 10% polyacrylamide gels (NuPage, Invitrogen, Grand Island, N.Y.) were equiloaded, electrophoresed at 200 V, electrotransferred to PVDF membranes, and probed with monoclonal antibodies to β-actin, CyclinD1, HGF, Notch-1, STAT3, p-STAT3, Erk1/2, p-Erk1/2 and p-p38 (all Abcam). Blots were developed by ECL (Thermo Scientific, Asheville, N.C.).

Polymerase Chain Reaction

For PCR analysis, total RNA was isolated using RNEasy Mini Kit (Qiagen, Germantown, Md.) and cDNA was made using the High Capacity Reverse Transcription kit (Applied Biosystems, Grand Island, N.Y.). RT-PCR was then performed, on a Stratagene Mx3005p (Promega, Madison, Wis.), using primers shown in Table 1:

TABLE 1

| Specificity | Primer | Sequence | Sequence ID |
|---|---|---|---|
| β-Actin | Forward | GGCTGTATTCCCCTCCATCG | SEQ ID NO: 1 |
| | Reverse | CCAGTTGGTAACAATGCCATGT | SEQ ID NO: 2 |
| c-fos | Forward | ATGTTGCGGTCGCTACGTC | SEQ ID NO: 3 |
| | Reverse | AGAAGTTGCCACCGCCG | SEQ ID NO: 4 |
| c-jun | Forward | CGATGCCCTCAACGCC | SEQ ID NO: 5 |
| | Reverse | CTTAGGGTTACTGTAGCCGTAGGC | SEQ ID NO: 6 |
| c-myc | Forward | ATGTTGCGGTCGCTACGTC | SEQ ID NO: 7 |
| | Reverse | AGAAGTTGCCACCGCCG | SEQ ID NO: 8 |
| Cyclin D1 | Forward | CGTACCCTGACACCAATCTC | SEQ ID NO: 9 |
| | Reverse | CTCCTCTTCGCACTTCTGCTC | SEQ ID NO: 10 |
| IL-4 | Forward | GGTCTCAACCCCCAGCTAGT | SEQ ID NO: 11 |
| | Reverse | GCCGATGATCTCTCTCAAGTGAT | SEQ ID NO: 12 |
| IL-22 | Forward | ATGAGTTTTTCCCTTATGGGGAC | SEQ ID NO: 13 |
| | Reverse | GCTGGAAGTTGGACACCTCAA | SEQ ID NO: 14 |
| IL-17 | Forward | TTTAACTCCCTTGGCGCAAAA | SEQ ID NO: 15 |
| | Reverse | CTTTCCCTCCGCATTGACAC | SEQ ID NO: 16 |
| IL-6 | Forward | TAGTCCTTCCTACCCCAATTTCC | SEQ ID NO: 17 |
| | Reverse | TTGGTCCTTAGCCACTCCTTC | SEQ ID NO: 18 |
| IFNγ | Forward | ATGAACGCTACACACTGCATC | SEQ ID NO: 19 |
| | Reverse | CCATCCTTTTGCCAGTTCCTC | SEQ ID NO: 20 |

The methods described used the techniques described in the following references, which methods are incorporated herein by reference. Koenecke C et al., European journal of immunology 2009; 39:372-9; Bedrosian A S et al., Gastroenterology 2011; 141:1915-26 e1-14; Wolf J H, et al., Liver transplantation: official publication of the American Association for the Study of Liver Diseases and the International Liver Transplantation Society 2014; 20:376-85; and Rehman A et al., J Immunol 2013; 190:4640-9

Results

Liver γδT Cells Express IL-17, IL-22, and Dectin-1 in Both Mice and Humans

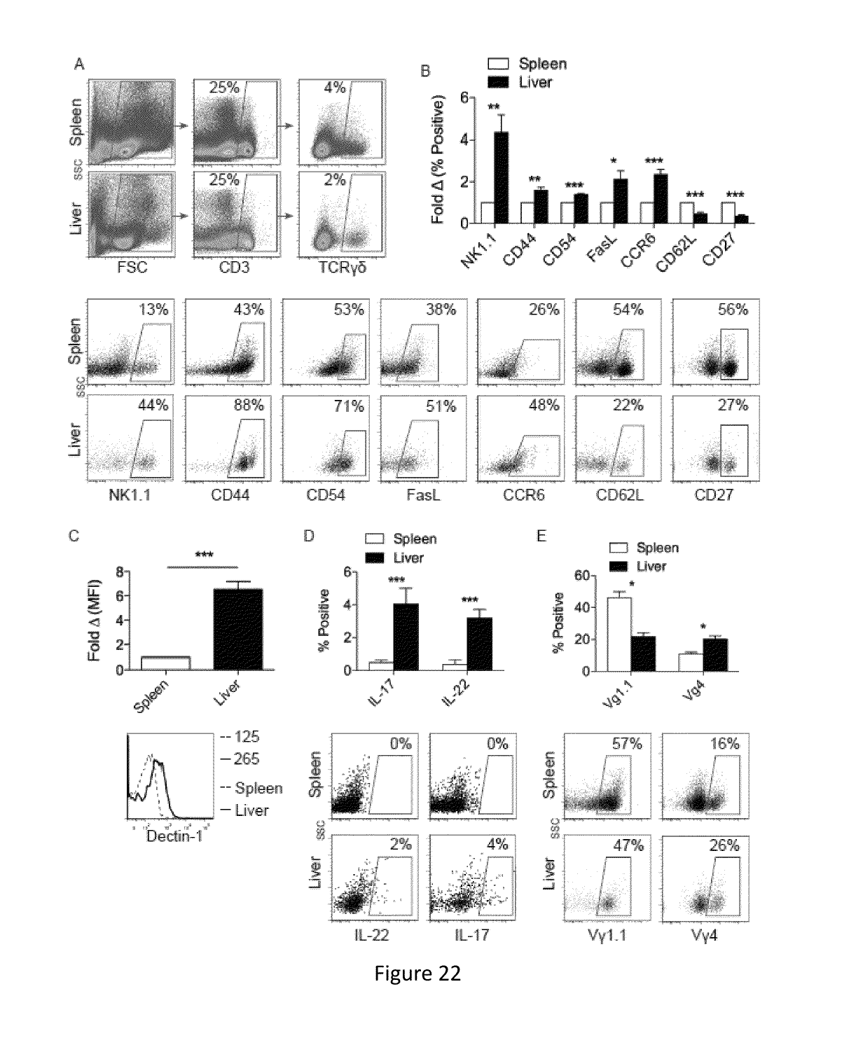

Before investigating the phenotypic and functional alterations in liver γδT cells during hepatic regeneration, we established their baseline characteristics. Compared with γδT cells in the spleen, liver γδT cells (FIG. 22A) were more activated, expressing elevated NK1.1, CD44, CD54, FasL, CCR6 and lower CD62L (FIG. 22B). Conversely, the CD27⁺ γδT cell population was more prominent in the spleen (FIG. 22B). Liver γδT cells also expressed higher IL-17 and IL-22 than their splenic counterparts (FIG. 22C) and higher Dectin-1 (FIG. 22D). Further, the hepatic γδT cell population contained a higher fraction of Vγ4⁺ cells, which are associated with enhanced functional activation, whereas Vγ1.1⁺ γδT cells predominated in the spleen (FIG. 22E).

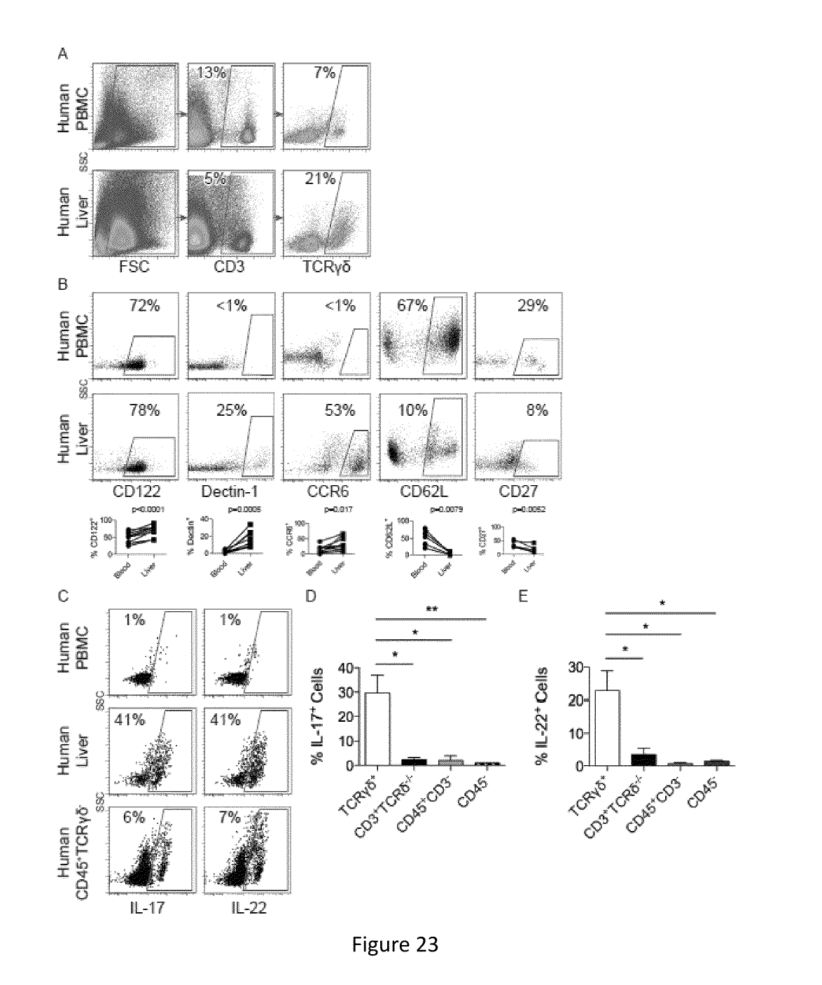

We observed parallel findings in the human liver, where hepatic γδT cells were more mature than their counterparts in the peripheral blood, expressing higher CD122, Dectin-1, and CCR6, as well as down-regulating CD62L and CD27 (FIG. 23A,B). Further, similar to their murine counterparts, human liver γδT cells produced markedly high levels of IL-17 and IL-22 relative to γδT cells in PBMC and compared to other hepatic parenchymal and inflammatory cells (FIG. 23C-E).

γδT Cells Expand after Partial Hepatectomy and are Necessary for Robust Regeneration

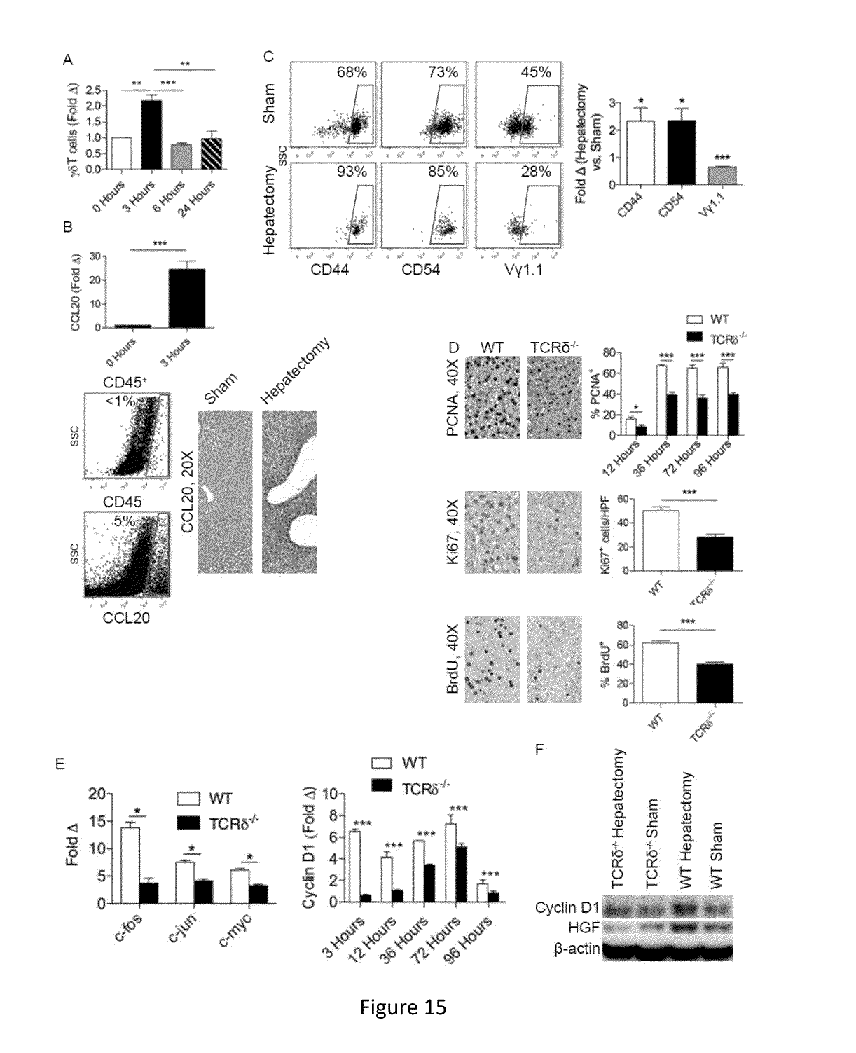

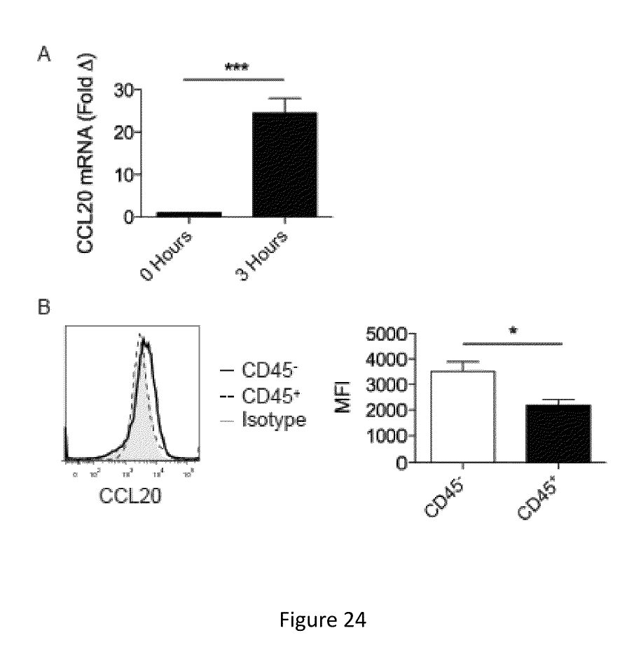

To investigate a potential role for γδT cells in liver regeneration, we determined whether this population was altered after partial hepatectomy. The number of intra-hepatic γδT cells increased sharply within the first 3 h after hepatectomy, but returned to baseline by 6 h (FIG. 15A). Correspondingly, there was a marked increase in hepatic expression of CCL20, a potent γδT cell chemokine, immediately following hepatectomy on analysis by PCR, immunofluorescence, and flow cytometry (FIGS. 15B, 24B). CCL20 was expressed mostly from hepatic parencymal cells (FIG. 15B, 24B). In addition, liver regeneration was associated with further γδT cell activation, as their population expressed higher CD44, CD54, and lower Vγ1.1 as compared to hepatic γδT cells from control liver (FIG. 15C).

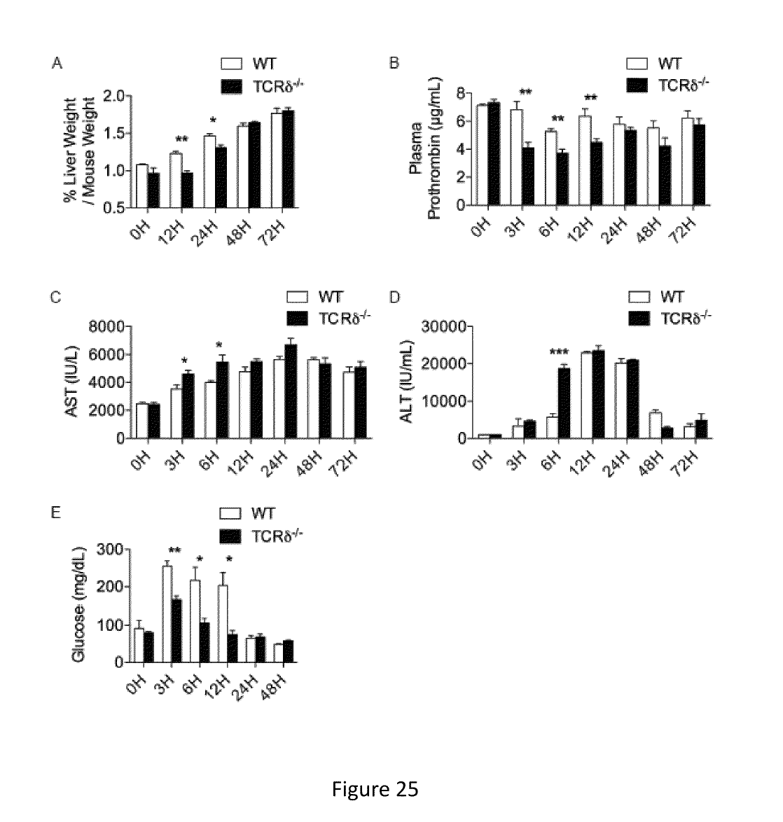

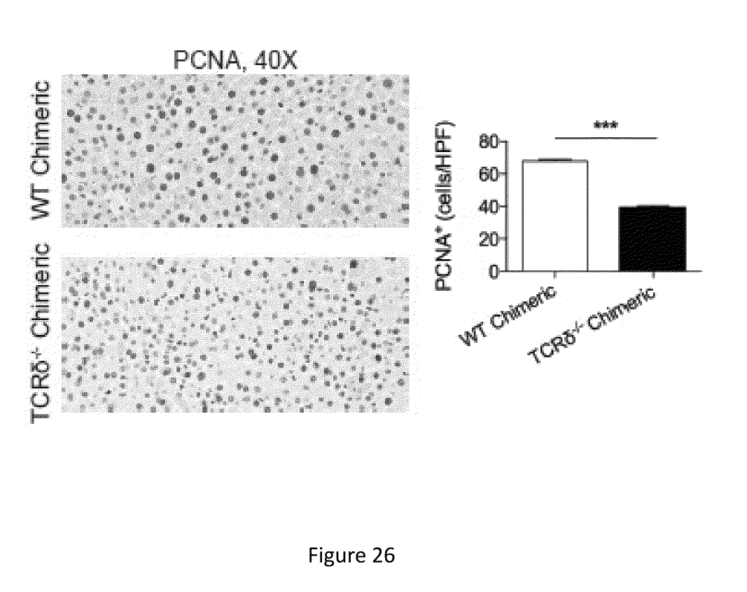

Since γδT cell populations expand and mature after partial hepatectomy, we postulated that they promote regeneration. To test this, we investigated the rate of liver regeneration in TCRδ^{-/-} mice. Hepatocyte proliferation was markedly lower in TCRδ^{-/-} animals compared to WT mice following partial hepatectomy, as measured by multiple proliferative indices including PCNA, Ki67, and BrdU incorporation (FIG. 15D). These data suggest that γδT cells are necessary for vigorous hepatic regeneration. Accordingly, expression of immediate early genes including c-fos, c-jun, and c-myc, was markedly higher in WT liver compared with TCRδ^{-/-} liver within the first hour after hepatectomy (FIG. 15E). Similarly, Cyclin D1 was expressed at substantially higher levels in WT liver compared to TCRδ^{-/-} liver at multiple post-hepatectomy time points (FIG. 15E,F). Elevations in hepatic expression of hepatocyte growth factor (HGF) were also depressed in absence of γδT cells (FIG. 15F). Consistent with a depressed rate of regeneration, weight of liver remnant and plasma levels of prothrombin were lower in TCRδ^{-/-} animals compared with WT at early time points after hepatectomy (FIG. 25A,B). WT mice also exhibited lower elevations serum transaminases but elevated serum glucose (FIG. 25C-E). WT mice made chimeric using bone marrow derived from TCRδ^{-/-} animals similarly exhibited a diminished rate of liver regeneration compared with WT mice transferred with WT bone marrow (FIG. 26).

Regenerating TCRδ^{-/-} Liver have a Dampened Inflammatory Milieu

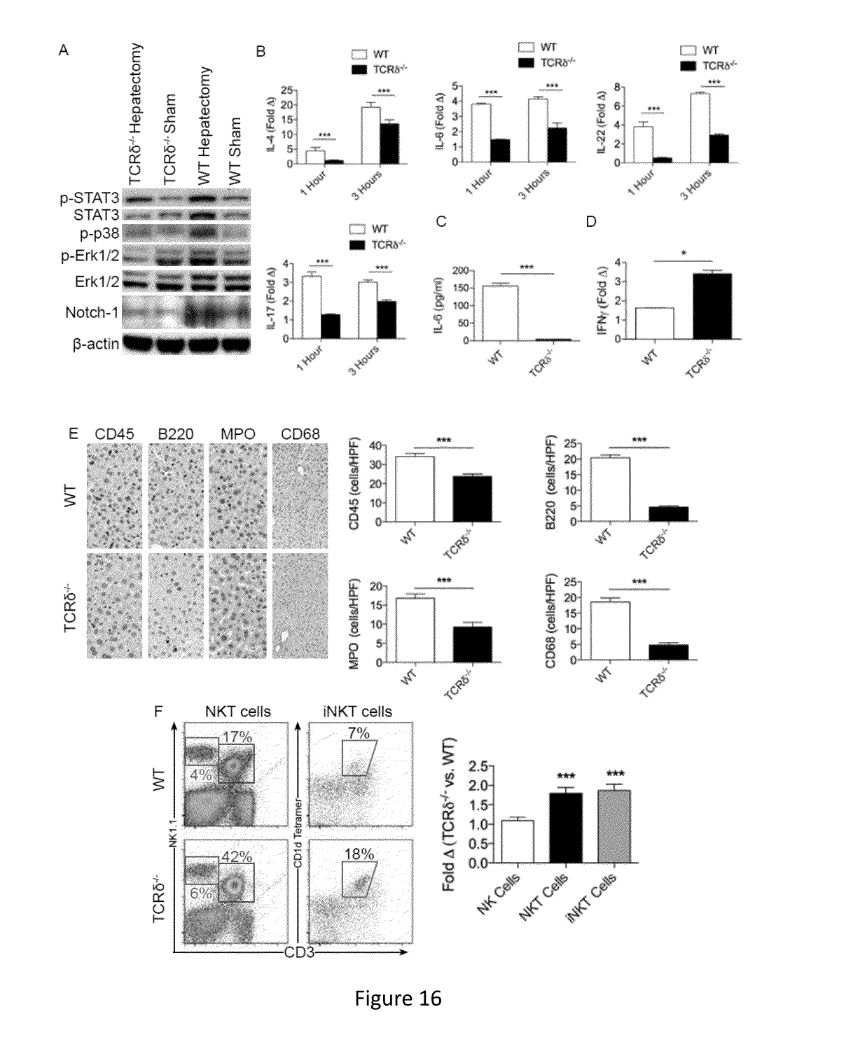

A dynamic interplay between inflammatory cytokines and pro-proliferative signaling pathways promote hepatic regeneration¹. To further investigate the mechanistic role of γδT cells in regeneration, we studied inflammatory signaling in regenerating TCRδ^{-/-} liver compared with WT. Liver from TCRδ^{-/-} mice expressed lower STAT3 and pSTAT3 and exhibited diminished phosphorylation of MAP kinase signaling intermediates within the first 3 h after partial hepatectomy (FIG. 16A). Similarly, there was absent elevation of Notch-1 expression in liver of regenerating TCRδ^{-/-} mice (FIG. 16A). There was also dampened elevation in hepatic expression of the pro-regenerative cytokines IL-4, IL-6, IL-22, and IL-17 after partial hepatectomy in TCRδ^{-/-} mice (FIG. 16B). Consistent with these findings, serum levels of IL-6 were markedly higher in hepatectomized WT mice compared with TCRδ^{-/-} (FIG. 16C). Conversely, IFNγ, which is associated with inhibited hepatocyte proliferation, was expressed at higher levels in TCRδ^{-/-} liver after hepatectomy (FIG. 16D). Taken together, these data suggest that the absence of γδT cells after partial hepatectomy is associated with muted expression of soluble inflammatory mediators and signaling intermediates that promote hepatic regeneration, along with elevated expression of inhibitory cytokines.

γδT Cells Regulate Intra-Hepatic Inflammatory Cell Recruitment During Liver Regeneration.

Based on our data illuminating the muted inflammatory milieu in regenerating TCRδ^{-/-} liver, we postulated that γδT cells may affect liver regeneration by influencing the recruitment and activation of neighboring immune subsets. The influx of bulk CD45⁺ hepatic leukocytes was markedly diminished in TCRδ^{-/-} mice after partial hepatectomy (FIG. 16E). Similarly, the numbers of B220⁺ cells, MPO⁺ cells, and CD68⁺ cells—which have been reported to contribute to hepatic regeneration—were lower in the livers of regenerating TCRδ^{-/-} mice compared with WT (FIG. 16E). Conversely, the fractions of bulk NKT cells and iNKT cells—whose activation has been associated with inhibiting hepatic regeneration when activated—were higher in TCRδ^{-/-} liver (FIG. 16E). Taken together, these data suggest that γδT cells are required for the generation of a pro-regenerative intra-hepatic leukocyte composition, whereas absence of γδT cells results in a diminished influx of neutrophils, B cells, and Kupffer cells, paired with an expansion of NKT cell populations.

γδT Cells Affect Inflammatory Cell Activation.

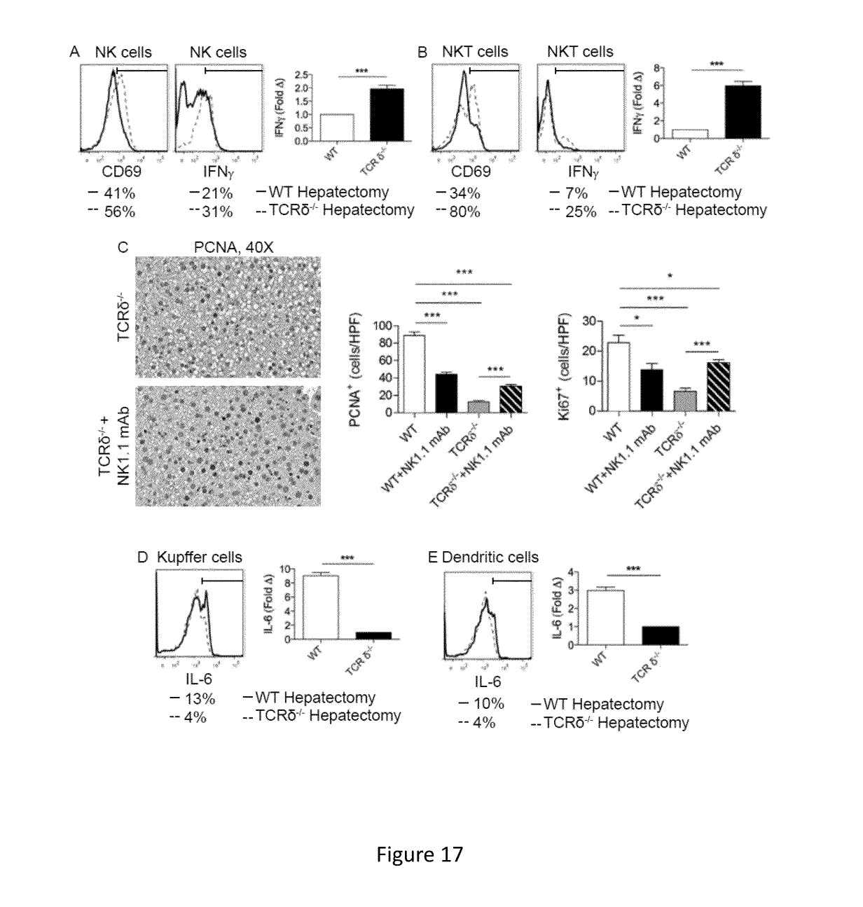

We postulated that γδT cells may affect liver regeneration by not only affecting leukocyte recruitment, but also influencing their activation. Using WT and TCRδ^{-/-} mice, we tested the in vivo phenotypic activation of intra-hepatic leukocyte subsets with established roles in modulating liver regeneration. We found that NK and NKT cells were more activated in regenerating TCRδ^{-/-} liver compared with WT, expressing elevated CD69 and producing higher IFN-γ (FIG. 17A,B). To investigate whether the presence of activated NK or NKT cells in TCRδ^{-/-} mice definitively contributed to their retarded liver regeneration, we selectively depleted these cellular subsets using a mAb directed against NK1.1. Depletion of NK and NKT cells partially reversed the depressed rate of liver regeneration in TCRδ^{-/-} mice (FIG. 17C). Interestingly, regeneration was depressed in WT mice following depletion of non-activated NK and NKT cell populations (FIG. 17C), a finding consistent with a recent report suggesting that NK and NKT cells can accelerate hepatic regeneration by upregulating IL-6 and HGF. Further, Kupffer cells and Dendritic cells—which are pro-regenerative—expressed higher IL-6 in regenerating WT liver as compared to TCRδ^{-/-} liver (FIG. 17D,E). Taken together, these data suggest that the presence of γδT cells affects the activation of varied inflammatory cell subsets with critical roles in modulating liver regeneration.

γδT Cells Influence the Activation of Hepatic Leukocyte Subsets Via IL-17

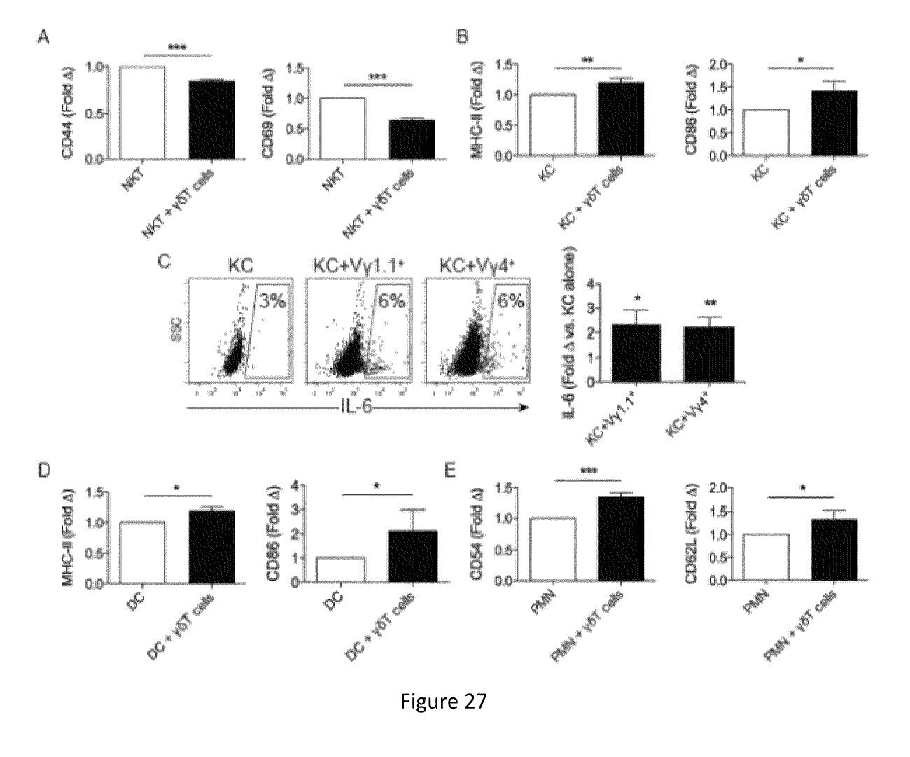

To test whether hepatic γδT cells can directly induce a pro-regenerative phenotype in neighboring hepatic leukocytes, we performed in vitro co-culture experiments. Hepatic γδT cells were purified by FACS and co-cultured with equal numbers of NKT cells, Kupffer cells, DC, or neutrophils. Consistent with our in vivo data, γδT cells induced diminished activation of NKT cells, modestly lowering their expression of CD44 and CD69 (FIG. 27A). Conversely, hepatic γδT cells modestly up-regulated expression of MHCII and CD86 on Kupffer cells (FIG. 27B), and induced their production of IL-6 (FIG. 27C). Both Vγ1.1⁺ and Vγ4⁺ subsets were equally effective activators of Kupffer cells (FIG. 27C). Similarly, γδT cells moderately activated the surface phenotype of DC (FIG. 27D) and neutrophils (FIG. 27E). Taken together, these data suggest that liver γδT cells can directly influence the generation of a pro-regenerative phenotype in neighboring hepatic innate inflammatory subsets.

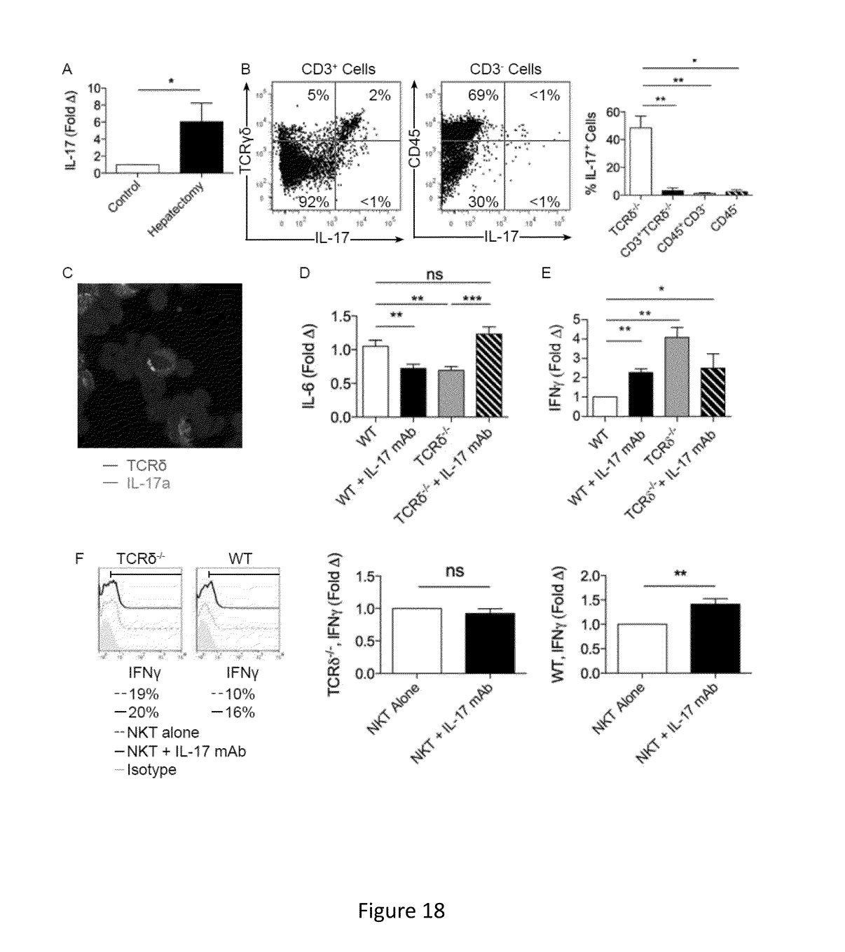

We found that hepatic γδT cells express elevated IL-17 at baseline in mice (FIG. 22D) and humans (FIG. 23C-E) which further increased markedly within 3 h following hepatectomy (FIG. 18A). Moreover, compared with hepatic CD3+TCRδ^{-/-} T cell subsets, CD3-CD45⁺ cells, and CD45− cells, a higher proportion of γδT cells were IL-17⁺ cells by flow cytometry in human liver (FIG. 23C,D) and in the regenerating mouse liver (FIG. 18B,C). Since emerging data suggest that IL-17 can modulate intra-hepatic sterile inflammation, we postulated that γδT cells induce a pro-regenerative hepatic inflammatory milieu via IL-17. To test this, leukocytes from WT mice and TCRδ^{-/-} mice were stimulated with PMA and ionomycin, in the presence or absence of an IL-17 mAb. WT leukocytes expressed higher levels of IL-6 at baseline compared with those from TCRδ^{-/-} mice (FIG. 18D), consistent with our previous observations that γδT cells promote the production of pro-regenerative cytokines. Moreover, WT leukocyte concentrates down-regulated IL-6 transcript in the context of IL-17 inhibition, whereas TCRδ^{-/-} leukocyte suspensions upregulated IL-6 production after IL-17 blockade (FIG. 18D). Further, in concert with our previous experiments, WT leukocyte concentrates expressed lower IFNγ compared with inflammatory cell suspensions from TCRδ^{-/-} mice (FIG. 18E). Conversely, IFNγ mRNA was upregulated after IL-17 blockade in WT leukocyte concentrates, but not in inflammatory cell concentrates from TCRδ^{-/-} mice (FIG. 18E). Taken together, these data suggest that IL-17 promotes inflammatory cell expression of high IL-6 and low IFNγ in γδT cell-rich leukocyte concentrates, but has the opposite effects in the absence of γδT cells.

To specifically investigate whether γδT cell-derived IL-17 influences the generation of a pro-regenerative NKT cell phenotype, we examined NKT populations in inflammatory cell suspensions derived from WT and TCRδ^{-/-} mice. We found that NKT cells produced higher IFNγ in the TCRδ^{-/-} suspensions (FIG. 18F), consistent with our in vivo observations of greater NKT cell activation in regenerating TCRδ^{-/-} liver (FIG. 17B). Additionally, blockade of IL-17 increased NKT cell IFNγ expression in γδT cell-rich cultures, but not in suspensions deficient in γδT cells (FIG. 18F). These data suggest that γδT cells can inhibit NKT cell activation via IL-17.

IL-17 Producing γδT Cells are Necessary for Robust Liver Regeneration.

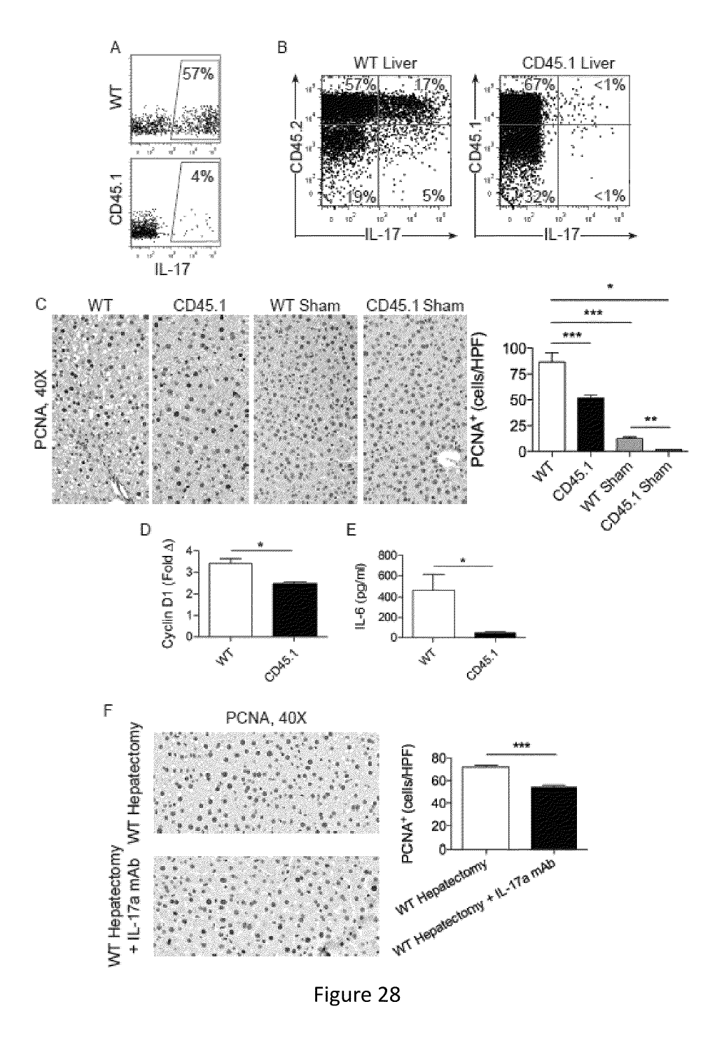

The CD45.1⁺ congenic C57BL/6 mouse sub-strain is genetically identical to the WT strain, except that it carries the differential B cell antigen Ly5.1. However, a recent report found that CD45.1⁺ mice exhibit selective deficiency in IL-17 producing γδT cells due to a mutation in Sox13 resulting in their defective development in the neonatal thymus. We confirmed markedly lower hepatic γδT cell expression of IL-17 in regenerating CD45.1 mice (FIG. 28A). Further, we showed that—consistent with absence of IL-17 producing γδT cells—there was almost no IL-17 production in regenerating liver of CD45.1 mice compared with WT (FIG. 28B). Therefore, we postulated that CD45.1 mice would have deficient hepatic regeneration. We found that CD45.1 mice exhibited a diminished rate of hepatocyte proliferation after 70% hepatectomy (FIG. 28C), reduced elevation in Cyclin D1 expression (FIG. 28D), and lower serum levels of IL-6 (FIG. 28E). Similarly, administration of a neutralizing IL-17 mAb to WT mice lowered the rate of hepatic regeneration (FIG. 28F).

γδT Cells Accelerate Liver Regeneration by IL-22 Production Via Dectin-1.

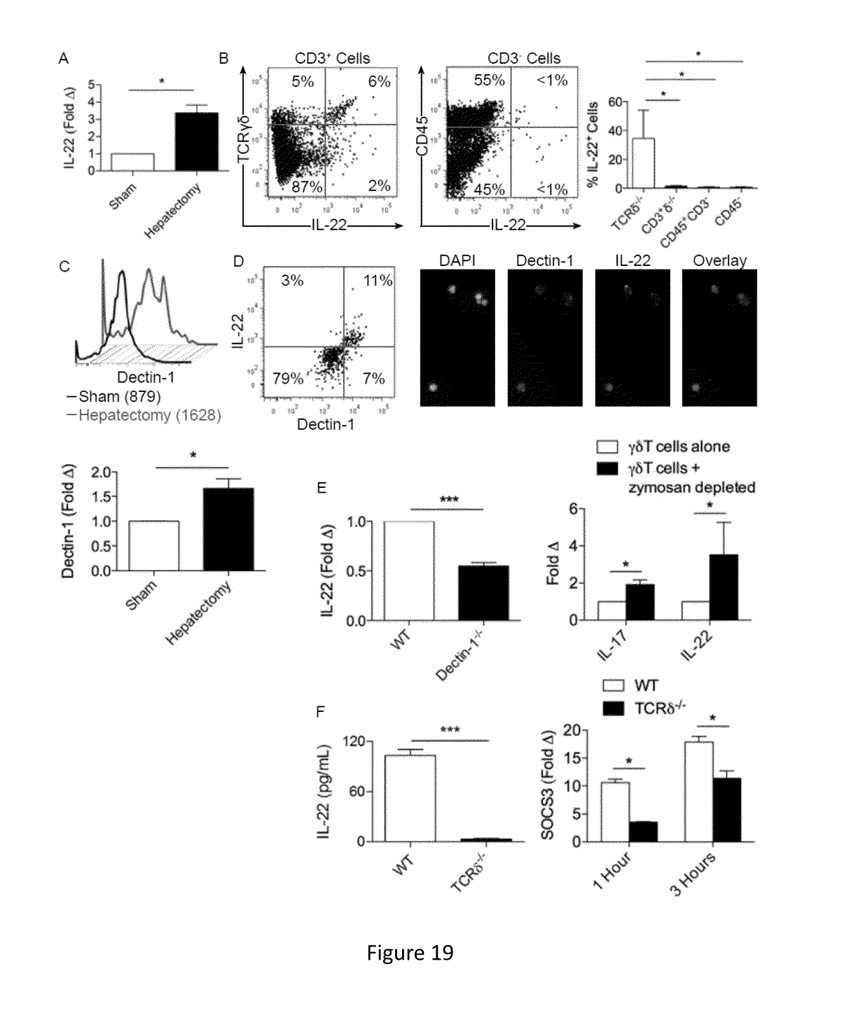

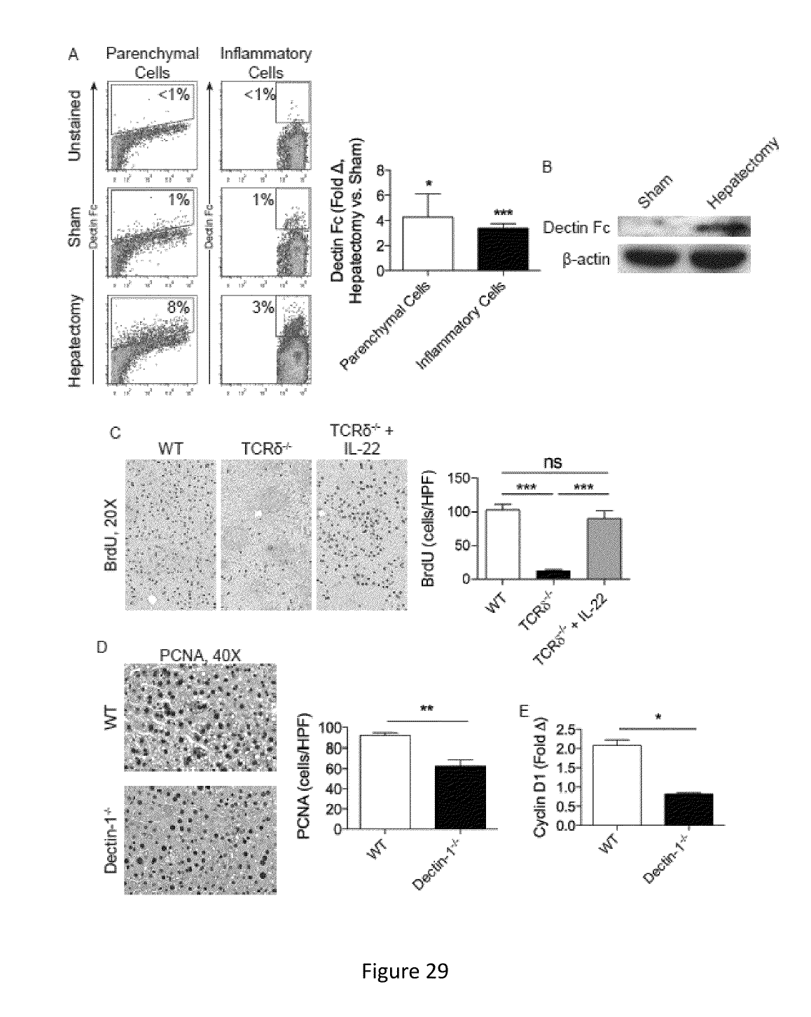

IL-22 is an IL-17-family cytokine that directly stimulates hepatocyte proliferation by inducing MAP kinase signaling. Innate inflammatory cells reportedly generate IL-22 in a Dectin-1 dependent manner. Therefore, we postulated that hepatic γδT cells, which express markedly elevated Dectin-1 (FIGS. 22C, 23B), influence hepatic regeneration via Dectin-1-dependent IL-22 production. We found that IL-22 expression was upregulated in hepatic γδT cells in the regenerating liver (FIG. 19A). Further, in parallel with IL-17, compared with hepatic CD3+TCRδ^{-/-} T cell subsets, CD3-CD45⁺ cells, and CD45− cells, a higher proportion of γδT cells were IL-22⁺ cells by flow cytometry (FIG. 19B). Dectin-1 expression was also upregulated on γδT cells in the regenerating liver compared with control liver (FIG. 19C). Co-expression analysis by both flow cytometry and confocal microscopy revealed that IL-22 was specifically expressed on Dectin-1⁺γδT cells (FIG. 19D) whereas hepatic γδT cells from Dectin-1^{-/-} mice exhibited an approximate 50% reduction in IL-22 production (FIG. 19E). Moreover, Dectin-1 ligation induced IL-22 and IL-17 production in hepatic γδT cells (FIG. 5E). In addition, using a human IgG Fc-Dectin-1-conjugated fusion protein, we found that hepatocytes and inflammatory cells over-express Dectin-1 ligands after partial hepatectomy (FIG. 29A, B). Further, the marked elevations in IL-22 expression associated with liver regeneration were diminished in TCRδ^{-/-} liver (FIG. 19F). Similarly, SOCS3 expression was lower in hepatectomized TCRδ^{-/-} mice compared with WT, consistent with reduced IL-22 signaling in the absence of γδT cells (FIG. 19F).

To test whether IL-22 production plays a primary role in the capacity of γδT cells to accelerate liver regeneration, we administered recombinant mIL-22 to TCRδ^{-/-} mice coincident with partial hepatectomy. As predicted, IL-22 administration rescued the depressed hepatic regeneration in TCRδ^{-/-} mice (FIG. 29C). Further, in concert with the dependence of IL-22 production on Dectin-1, we found retarded rates of liver regeneration in Dectin-1^{-/-} mice following partial hepatectomy along with a concomitant diminished upregulation of Cyclin D1 (FIG. 29D, E).

The Dectin-1-γδT Cell-IL-17 Axis Regulates Liver Regeneration

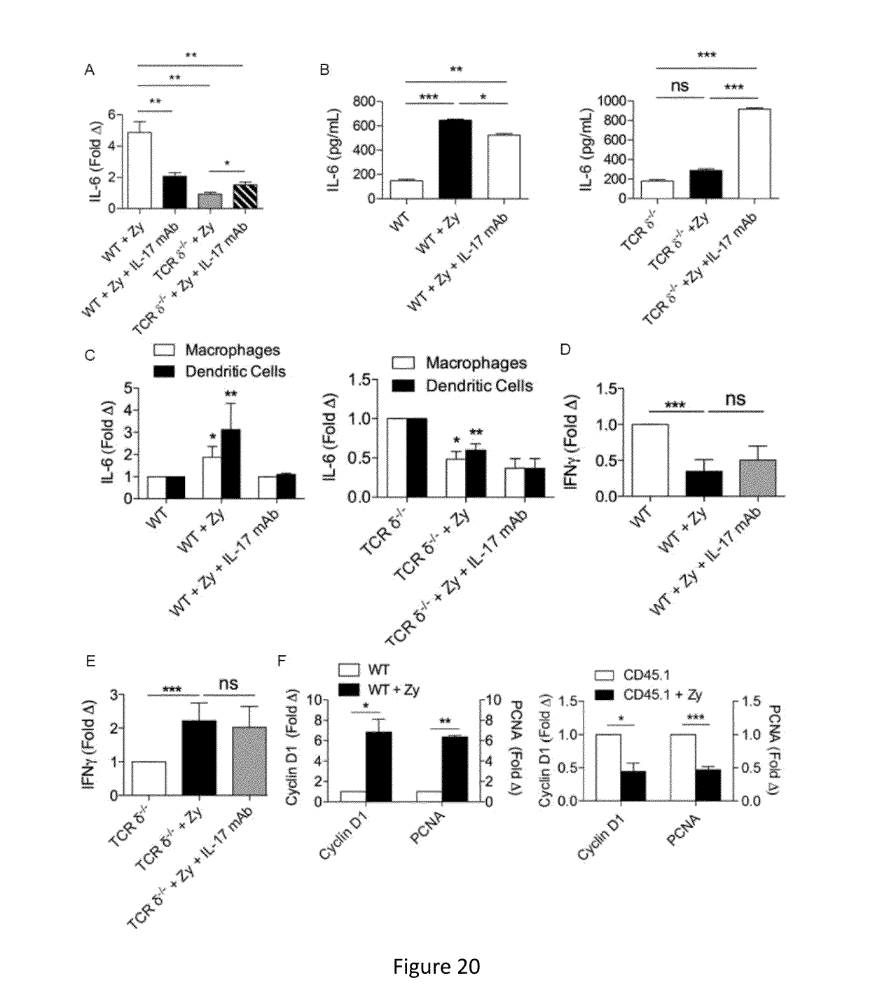

To further investigate the link between Dectin-1-mediated IL-17-family cytokine production and γδT cell induction of pro-regenerative leukocytes, we stimulated leukocyte suspensions from WT or TCRδ^{-/-} mice with Zymosan depleted (Zy) to selectively activate Dectin-1 signaling. WT leukocyte concentrates produced higher IL-6 in response to Zy stimulation, as compared to γδT cell-depleted leukocyte suspensions (FIG. 20A). Moreover, IL-17 blockade diminished the activation of Zy-stimulated leukocytes in the presence of γδT cells, but had the opposite effect in their absence, as measured by both mRNA (FIG. 20A) and protein analyses (FIG. 20B). To determine whether Dectin-1 activation of γδT cells specifically results in a pro-regenerative phenotype in macrophages and DC in an IL-17-dependent manner, we gated on these cellular subsets in Zy-stimulated γδT cell-rich and γδT cell-depleted leukocyte concentrates and measured their production of IL-6 by intracellular cytokine analysis. In γδT cell-rich suspensions, both macrophages and DC upregulated expression of IL-6 in response to Dectin-1 ligation, the effect of which was abrogated by an IL-17 mAb (FIG. 20C). Conversely, macrophages and DC did not become activated by Zy in TCRδ^{-/-} leukocyte concentrates (FIG. 20C). By gating on NKT cells and measuring IFNγ expression, we further found that NKT cells markedly diminished their production of IFNγ in response to Zy in WT cultures (FIG. 20D). Conversely, in TCRδ^{-/-} cultures, NKT cells increased IFNγ production in response to Dectin-1 ligand (FIG. 20E). Taken together, these data show that Dectin-1-mediated IL-17-dependent inflammatory responses, and specifically their induction of a pro-regenerative phenotype in neighboring leukocyte subsets, is contingent on the presence of γδT cells.

To directly test the central role of the Dectin-1-γδT cell-IL-17 axis in vivo in the regenerating liver, we administered Zy to WT and CD45.1 mice coincident with partial hepatectomy. We found that Dectin-1 ligation markedly increased Cyclin D1 expression in the liver of regenerating WT mice, but lowered Cyclin D1 expression in the CD45.1 liver (FIG. 20F). Similarly, hepatocyte proliferation was accelerated in WT mice but diminished in CD45.1 mice following Zy administration (FIG. 20F), consistent with our observation of paradoxical anti-inflammatory and anti-regenerative effects of Dectin-1 ligation in the absence of IL-17-producing γδT cells.

### DISCUSSION

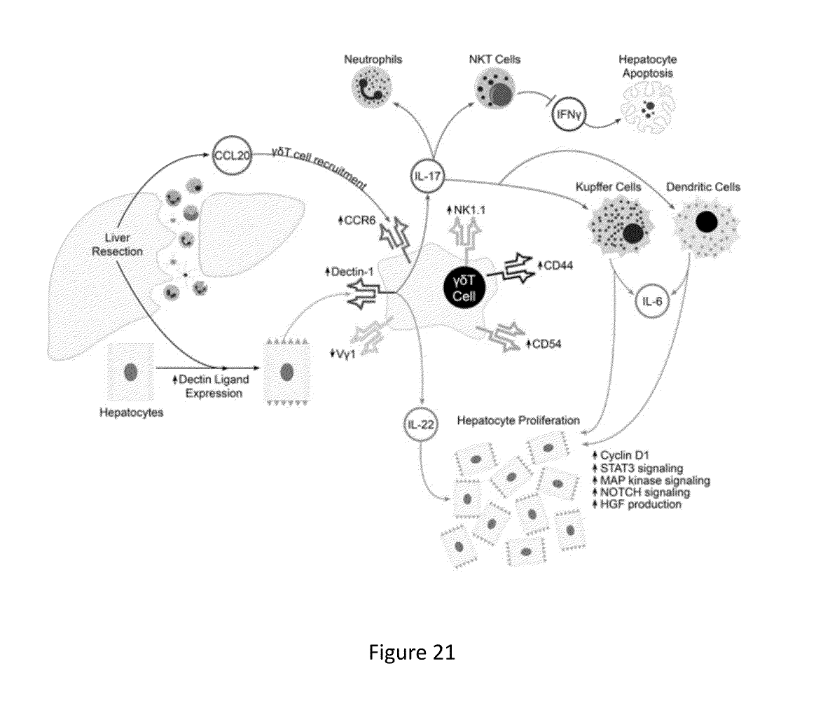

Our work reveals a novel role for IL-17/IL-22-producing γδT cells in governing the inflammatory orchestration of hepatic regeneration by regulating the phenotype and recruitment of diverse hepatic leukocytes (FIG. 21). While our correlative data in human systems suggests relative activation of human liver γδT cells and production of IL-17 family cytokines, the nomenclature and complexity of γδT cell subsets vary from mice to humans as do the specific γδT cell receptors for activating ligands suggesting caution before translating our data directly to human hepatic regeneration.

We found that Dectin-1 ligands are highly expressed on both parenchymal and inflammatory cells within hours after partial hepatectomy, and exogenous Dectin-1 ligand administration accelerates hepatocyte priming in a γδT cell- and IL-17-contingent manner. A report showed for the first time that intermediate filaments such as vimentin can ligate and activate Dectin-1. Our data suggests that Dectin-1 activation can be used to promote hepatic regeneration after surgical resection or for patients with acute or chronic liver disease.

Downstream of Dectin-1, we ascribe a central role for IL-17-family cytokines in liver regeneration. We found that CD45.1 mice, whose γδT cells have diminished ability to produce IL-17, exhibit depressed liver regeneration and response to Dectin-1 ligation. Whereas CD4⁺ T helper cell subsets have frequently been considered primary sources of IL-17-related cytokines in neoplastic and inflammatory settings, we found minimal IL-22 or IL-17 secretion from CD3+TCRδ^{-/-} T cells after hepatectomy, while γδT cells robustly produced both cytokines in the regenerating liver.

Our experimental results suggest that IL-17 produced by γδT cells is critical in inducing a pro-regenerative phenotype in hepatic inflammatory cells including Kupffer cells, DC, and neutrophils, while simultaneously inhibiting NKT cell expansion or activation. Indeed, NKT cells are critical cellular targets of the Dectin-1-γδT cell-IL-17 axis within the regenerating liver. This is exemplified by our findings that depletion of NKT cells in vivo accelerates liver regeneration and hepatocyte expression of Cyclin D1 in TCRδ^{-/-} mice, yet has the inverse effect of slowing liver regeneration in WT mice. This paradoxical role for NKT cells in liver regeneration—contingent on both their activation and their interaction with γδT cells—is consistent with recent reports showing that activated NKT cells which express IFNγ inhibit liver regeneration. Conversely, non-activated NK or NKT cells promote liver regeneration in WT mice⁴. It is conceivable that, in the presence of γδT cells, the reduced IFNγ synthesis from NKT cells shifts their functional properties from anti-regenerative to pro-regenerative owing to NKT cells simultaneously being sources of IL-4, a pro-regenerative cytokine.

We found that in addition to IL-17, γδT cells are a vital source of IL-22 in the regenerating liver. IL-22 can directly induce hepatocyte proliferation. However, the cellular sources of IL-22 during hepatic regeneration had not been previously demonstrated. Since innate inflammatory cells generate IL-22 in a Dectin-1 dependent manner, we suspected that hepatic γδT cells—which express markedly elevated Dectin-1—can influence hepatic regeneration via Dectin-1-dependent IL-22 production. We showed that IL-22 expression from γδT cells was Dectin-1 dependent. Further, administration of recombinant IL-22 impressively restored the sluggish rate of liver regeneration in TCRδ^{-/-} mice. IL-22 signals via the JAK/STAT pathway to upregulate numerous pro-regenerative genes, including TNFα and IL-6. Accordingly, we observed diminished elevations in SOCS3 and MAP kinase intermediates—which serve as surrogate markers of IL-22 signaling—in the regenerating liver of TCRδ^{-/-} mice. Our observations of Dectin-1-mediated IL-22 expression in liver regeneration parallels its effects in pathogenic contexts, where Dectin-1 ligation has been associated with anti-fungal immunity in the lung via IL-22 production.

A novel observation inherent to the mechanism underlying our in vivo findings, and emphasized by our in vitro studies, is that whereas IL-17 cytokine blockade in the presence of γδT cells has the effect of inhibiting a pro-regenerative phenotype in neighboring leukocyte subsets, including down-regulating IL-6 and upregulating IFNγ, it has diametrically opposite effects in the absence of γδT cells. These patterns are amplified by Dectin-1 ligation in both in vitro co-culture experiments and in our in vivo model of hepatic regeneration, suggesting that the IL-17-dependent inflammatory effects of Dectin-1 signaling are contingent on γδT cells. As such, we ascribe a novel role for the Dectin-1-γδT cell-IL-17 axis in promoting liver regeneration, characterized by the induction of a pro-regenerative inflammatory milieu following hepatectomy. Our work further suggests a role for Dectin-1 in sterile inflammation and may have broader implications to the importance of γδT cells in directing Dectin-1 mediated IL-17-dependent inflammation in both sterile and pathogenic contexts.

1. A method of treating a liver disorder in an individual comprising administering to the individual a therapeutically effective amount of an activator of Dectin-1 pathway.

2. The method of claim 1, wherein the liver disorder is sterile inflammation, sepsis, liver fibrosis, liver cirrhosis, and hepatocellular carcinoma.

3. The method of claim 1, wherein the activator of Dectin-1 pathway is a Dectin-1 ligand.

4. The method of claim 1, wherein the activator of Dectin-1 pathway is an inhibitor of CD14.

5. A method for identifying an agent which activates Dectin-1 pathway comprising exposing cells which express Dectin-1 to a test agent and determining if TLR4 activation and/or CD14 expression is decreased compared to the activation in the absence of the test agent.

6. The method of claim 5, wherein the cells expressing Dectin-1 are selected from the group consisting of dendritic cells, macrophages, CD14⁺ monocytic cells and hepatic stellate cells.

7. The method of claim 6, wherein the cells are macrophages.

8. The method of claim 7, further comprising the step of obtaining the macrophages from an individual.

9. The method of claim 6, further comprising obtaining whole blood from an individual and isolating a fraction enriched for macrophages from the whole blood, prior to exposing the macrophages to the test agent.
